(19) United States
(12) Patent Application Publication
(10) Pub. No.: US 2023/0230701 A1
(43) Pub. Date: Jul. 20, 2023

(54) Methods and Systems for Generating and Monitoring Holistic Treatment Processes

(71) Applicant: Vell LLC, Jupiter, FL (US)

(72) Inventors: Ken Grey, Jupiter, FL (US); Michael Gioan, Huntington Beach, CA (US)

(73) Assignee: Vell LLC, Jupiter, FL (US)

(21) Appl. No.: 18/099,809

(22) Filed: Jan. 20, 2023

Related U.S. Application Data
(60) Provisional application No. 63/301,446, filed on Jan. 20, 2022.

(51) Int. Cl. G16H 50/20; G16H 20/60; G16H 20/70; G16H 20/30; G16H 40/67

(52) U.S. Cl. CPC G16H 50/20; G16H 20/60; G16H 20/70; G16H 20/30; G16H 40/67

(57) ABSTRACT

Systems and method are provided for generating and monitoring holistic treatment process. A computing device may receive an identification of symptoms associated with a user profile. The computing device may execute a machine-learning model using the user profile and the symptoms to generate a holistic treatment process configured to alleviate the symptoms. The computing device may receive performance data corresponding to the execution of the holistic treatment process over a first time interval and, in response, modify the machine-learning model using the performance data to generate an updated machine-learning model. The updated machine-learning model may be configured to generate a revised holistic treatment process that is more likely to alleviate the one or more symptom. The computing device may then facilitate a presentation of the revised holistic treatment process.

### CROSS-REFERENCE TO RELATED APPLICATIONS

The present patent application claims the benefit of priority to U.S. Provisional Patent Application No. 63/301,446 filed Jan. 20, 2022, which is incorporated herein by reference in its entirety for all purposes.

### TECHNICAL FIELD

This disclosure relates generally to generating holistic treatment processes, and more particularly to generating and monitoring holistic treatment processes configured for distributed execution.

### BACKGROUND

Holistic medicinal processes attempt to alleviate conditions as an alternative to or an augment of traditional medical processes. Holistic medicinal assess and treat the body as an entire system rather than as individual components. For example, a migraine may be treated with analgesic medication, which can be replaced with and/or augmented by holistic treatment processes that may address the entire system such as, exercising the body, reducing stress through meditation or therapy, recommending changes in sleep patterns, changes in nutrition to reduce trigger foods that may cause or influence the cause of the migraines, etc.

Each component of the overall system may have subtle dependencies on other components of the system. The features that may cause one component to depend on another may be unknown. For example, adjusting one component can cause measurable changes in a corresponding component without identifying the mechanism of that may cause the measurable change. In some instances, a first adjustment to a component of the system may cause a measurable change in a corresponding (e.g., dependent) component, while a different adjustment may not cause a detectable change, obscuring both the dependency between components and the mechanism that may cause the dependency. In some instances, a first adjustment to a first component may negatively impact corresponding components, while another adjustment may cause improvements to the corresponding components and/or the overall system.

### SUMMARY

Methods are described herein for generating and monitoring holistic treatment processes. The method includes: receiving an identification of one or more symptoms, the one or more symptoms being associated with a user profile; executing a machine-learning model using the identification of the one or more symptoms and the user profile, the machine-learning model being configured to generate a holistic treatment process, wherein the holistic treatment process is configured to alleviate the one or more symptoms when executed by a user, and wherein the holistic treatment process includes treatment protocols for a set of interdependent holistic classes; facilitating a presentation of the holistic treatment process; receiving performance data corresponding to execution of the holistic treatment process over a first time interval; modifying the machine-learning model using the performance data to generate an updated machine-learning model, wherein the updated machine-learning model is configured to generate a revised holistic treatment process that is more likely to alleviate the one or more symptom; and facilitating a presentation of the revised holistic treatment process, wherein the revised holistic treatment process, when executed by the user, increases a likelihood of alleviating the one or more symptoms.

Systems are described herein for generating and monitoring holistic treatment processes. The systems include one or more processors and a non-transitory computer-readable medium storing instructions that, when executed by the one or more processors, cause the one or more processors to perform any of the methods as previously described.

A non-transitory computer-readable media described herein may store instructions which, when executed by one or more processors, cause the one or more processors to perform any of the methods as previously described.

These illustrative examples are mentioned not to limit or define the disclosure, but to aid understanding thereof. Additional embodiments are discussed in the Detailed Description, and further description is provided there.

### BRIEF DESCRIPTION OF THE DRAWINGS

Features, embodiments, and advantages of the present disclosure are better understood when the following Detailed Description is read with reference to the accompanying drawing.

FIG. 1 is a block diagram of an example holistic processing system according to aspects of the present disclosure.

FIG. 2 is a block diagram of an example communication network configured to facilitate access to operations of a holistic processing application according to aspects of the present disclosure.

FIG. 3 illustrates an example graphical user interface of a holistic processing application depicting a status of a user according to aspects of the present disclosure.

FIG. 4 illustrates an example graphical user interface of a holistic processing application depicting a status of a particular interdependent holistic class according to aspects of the present disclosure.

FIGS. 5A and 5B illustrate example graphical user interfaces of a holistic processing application depicting acquisition of information usable to define holistic treatment process and/or a status of a user according to aspects of the present disclosure.

FIG. 6 illustrates a flowchart of an example process for generating holistic treatment processes executable to improve an overall wellness of a particular user according to aspects of the present disclosure.

FIG. 7 illustrates an example graphical user interface of an application configured to remotely modify interfaces of a holistic processing application associated with interdependent holistic classes according to aspects of the present disclosure.

FIG. 8 illustrates an example graphical user interface of an application configured to remotely modify interfaces of a holistic processing application associated with symptoms according to aspects of the present disclosure.

FIG. 9 illustrates an example graphical user interface of an application configured to remotely modify interfaces of a holistic processing application associated with treatment processes according to aspects of the present disclosure.

FIG. 10 illustrates another example graphical user interface of an application configured to remotely modify interfaces of a holistic processing application associated with treatment processes according to aspects of the present disclosure.

FIG. 11 illustrates another example graphical user interface of an application configured to remotely modify interfaces of a holistic processing application defining treatment processes according to aspects of the present disclosure.

FIGS. 12A, 12B. 12C, and 12D illustrate example graphical user interfaces of a holistic processing application dynamically defined based remotely received instructions according to aspects of the present disclosure.

FIG. 13 illustrates a flowchart of an example process for generating holistic treatment processes executable to improve an overall wellness of a particular user according to aspects of the present disclosure.

FIG. 14 illustrates an example computing device architecture of an example computing device that can implement the various techniques described herein according to aspects of the present disclosure.

### DETAILED DESCRIPTION

Methods and systems are described herein for generating and monitoring holistic treatment processes. A holistic processing application may receive user data associated with a particular user. The user data may be received from user input, sensor data from one or more devices in communication with the device executing the holistic processing application, and/or data from one or more remote devices configured to execute treatment protocols or portions thereof for the particular user. The holistic processing application may define individual values for each of one or more interdependent holistic classes indicative a state of the particular user. The holistic processing application may also define an overall value from the values of the one or more interdependent holistic classes. The individual values and the overall value may be indicative of a wellness of the particular user.

The holistic processing application may enable selection of an interdependent holistic class and/or the overall value. In response, the holistic processing application may provide additional information associated with the selected interdependent holistic class or overall value such as, but not limited to: a history of the individual value or overall value over a selected time interval; one or more symptoms affecting the individual value or overall value; an identification of one or more interdependent holistic class that may be affecting the individual value, the overall value, and/or the selected interdependent holistic class; demographic information, a weight assigned to the selected interdependent holistic class, an identification of executable processes that may improve the individual value of the selected interdependent holistic class or overall value (e.g., such as links to component processors, videos, articles, associated interdependent holistic classes that may improve the selected interdependent holistic class, etc.); combinations thereof, or the like.

In one illustrative example, the holistic processing application may receive an identification of one or more symptoms associated with the user. The holistic processing application may generate a holistic treatment process based on the user information to alleviate the one or more symptoms using one or more machine-learning models (e.g., using neural network, ensemble models, etc.). The holistic treatment process may include one or more treatment protocols for each of one or more interdependent holistic classes. Each treatment protocol may be executed by the particular user, the user device executing the holistic processing application, and/or by one or more component processors (e.g., operating remote from the user device). The holistic treatment process may be associated with a time interval over which the holistic treatment process is to execute. The holistic processing application may monitor the execution of the holistic treatment process in real time as the holistic treatment process is executed over the time interval.

During the time interval and/or after, performance data associated with the execution of the holistic treatment process may be generated. The performance data may include feedback (e.g., from user input from the particular user, component processors, etc.), sensor data from the user device and/or devices in communication with the user device, execution related data (e.g., identifying any faults, interrupts, etc., detected in the execution of the holistic treatment process, determining whether the holistic treatment process terminated successfully, determining whether the holistic treatment process terminated within the time interval, etc.,), combinations thereof, or the like. The performance data may be used to modify the machine-learning model. The modified machine-learning model may be configured to generate revised holistic treatment processes for the particular user that may be more likely to alleviate the one or more symptoms of the user. The modified machine-learning model may be used to train other machine-learning models usable to generate holistic treatment processes for other users and/or user devices (e.g., such as those that have characteristics in common with the particular user and/or user device, etc.).

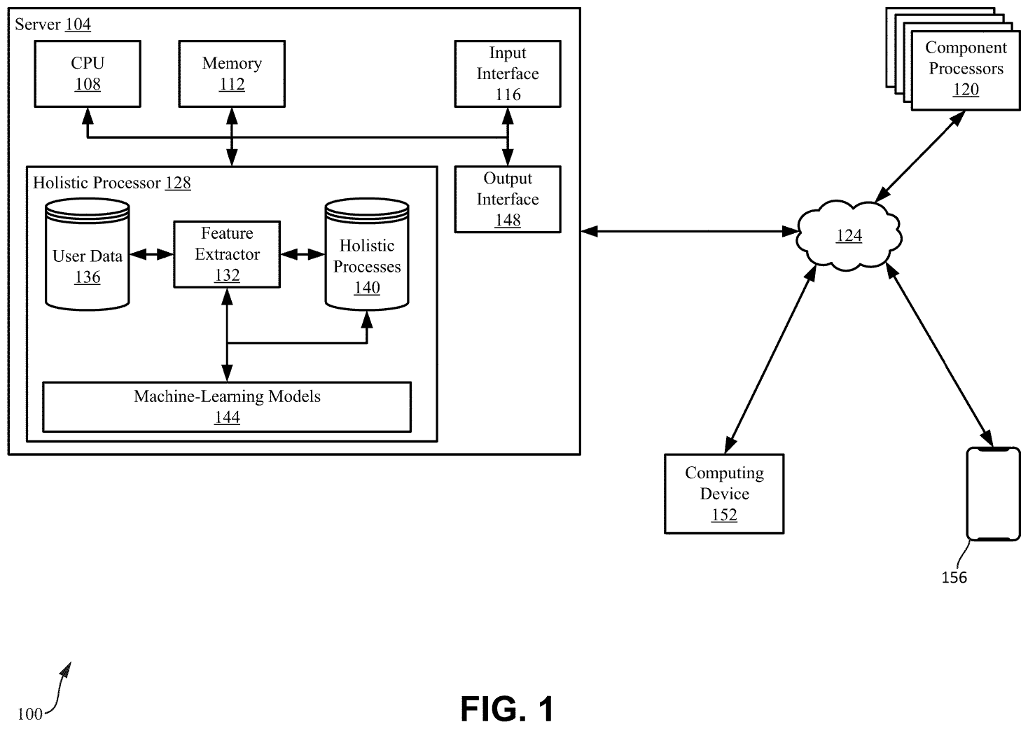

FIG. 1 is a block diagram of an example holistic processing system according to aspects of the present disclosure. Holistic processing system 100 may include hardware and/or software components that operate within a distributed environment to provide holistic treatment processes. For example, holistic processing system 100 may include servers (e.g., such as server 104) that may host applications and/or provide services accessible to user devices (e.g., computing device 152, mobile device 156, and/or the like), component processors 120, and/or other devices. In a distributed environment, some processes of the holistic processing system 100 (e.g., such as, but not limited to, the applications, services provided, and/or portions thereof) may be executed by one or more other devices such as, but not limited to, one or more user devices, one or more component processors 120, and/or one or more other devices (e.g., databases, servers, content providers, cloud networks, etc.). In some instances, holistic processing system 100 may execute within a single environment (e.g., such as in a client/server configuration) in which server 104 may host the applications and/or provide the services of the holistic processing system 100 to the user devices and/or component processors 120. The user devices and/or component processors 120 may execute applications and/or processes that access server 104 to enable generation of holistic treatment processes for users, monitor and/or manage holistic treatment processes, monitor and/or manage user wellness, etc.

Server 104 may include processing hardware (e.g., one or more processors such as CPU 108, memory 112, input interfaces 116, output interfaces 148, etc.) and holistic processor 128. Server 104 may be configured to host, manage, and/or provide services to a holistic processing application. In some instances, holistic processor 128 may include software instructions and/or hardware (e.g., processors, memory, field programmable gate arrays (FPGAs), etc.) that facilitate the execution of the holistic processing application. In those instances, CPU 108, memory 112, input interface 116, output interface 148 and holistic processor 128 may be connected via a bus or other physical data interconnection. In other instances, holistic processor may include software instructions configured for execution by CPU 108 and/or one or more remote devices (e.g., user devices and/or component processors 120, databases, other servers, etc.). In those instances, CPU 108, memory 112, input interface 116, output interface 148 may be connected the bus or other physical data interconnection. Holistic processor 128 may include software instructions that are stored in non-volatile memory (e.g., memory 112, one or more other local memories, one or more remote memories, and/or one or more local and remote memories, etc.). The non-volatile memory may be connected via the bus or other physical data interconnection. Alternatively, the non-volatile memory may be directly connected to CPU 108.

Holistic Processor 128 may include one or more databases that store information associated with users of holistic processing system 100 and information associated with component processors 120. For example, holistic processor 128 may include a first database (e.g., user data 136) that stores data associated with users. Holistic processor 128 may receive data associated with user directly from user devices operated by those users and from component processors 120 that may provide holistic treatment processes for the user devices. For example, a first user may download a holistic processing application and register with holistic processor 128. The holistic processing application may request information associated with a state of the user (e.g., to determine a baseline wellness and to determine areas in which the holistic processing system may provide holistic treatment processes to improve the baseline wellness, etc.). The information may include, but is not limited to, fitness and/or exercise information, information corresponding to current or historical treatments (holistic and/or medical), nutrition and food intake information, mental health information of the user, information of current or historical supplements taken by the user, information of symptoms and/or ailments of the user, information related to any diagnosis (e.g., by an expert system such as, but not limited to, medical or holistic professionals, etc.), demographic information (e.g., age, gender, ethnicity, etc.), location information, information associated with routines of the user, an identification of a profession of the user, information associated with pain experienced by the user, information associated with goals of the user, etc.

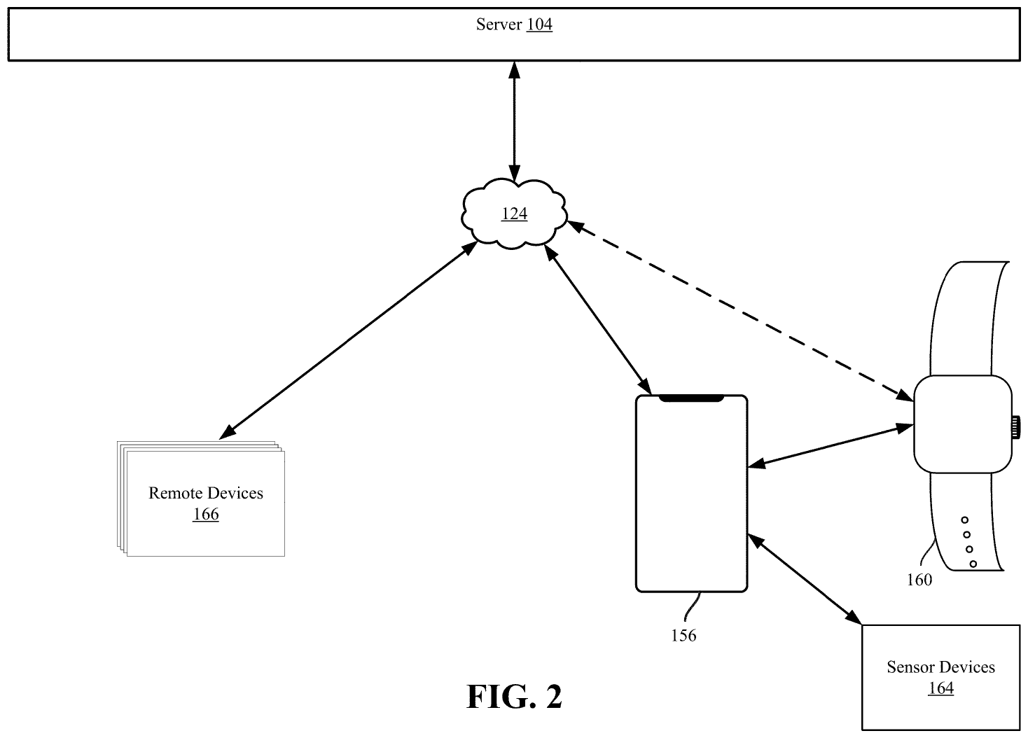

In some instances, holistic processor 128 may receive information from devices connected to user devices. User devices may include sensors or receive sensor data from sensor devices 164 such as, but not limited to, accelerometers, speedometers, electroencephalograms, electrocardiograms, pulse oximeters, sweat sensing devices, heart rate monitors, heart rate variability monitors, or the like. As shown in FIG. 2, user device 156 may be connected to smartwatch 160 and/or sensor devices 164 that can include a heart rate (and/or other sensors) that may transmit heart rate data associated with the user to user device 156. User device 156 may store the heart rate data and/or transmit the heart rate data for storage in user data 136.

Component processors 120 may also provide information associated with the user to holistic processor 128. Component processors 120 may include expert systems, devices, applications, etc. that provide services to users. For example, a first component processor 120 may include a mental health application that provides mediation services and/or other mental health services through media (e.g., audio and/or video), direct communications (e.g., audio and/or video), etc. The first component processor 120 may collect data associated with the user while providing services to the user. The first component processor 120 may transmit may store this data locally, transmit this data to the user device of the user, and/or transmit this data to user data 136.

Other databases of holistic processor 128 may include holistic treatment processes 140. Holistic treatment processes 140 may store holistic treatment process generated by machine-learning models 144 and/or the processes of the holistic processing application. Once generated, an identification of the holistic treatment process may be stored in holistic treatment processes 140 in associated with a user identifier of the user for which the holistic treatment process was generated, feedback from the user, information associated with the efficacy of the holistic treatment process (e.g., such as, but not limited to, an indication of a change in a status of the user, an indication of an increase or decrease in pain of the user, an indication of a change in the symptoms of the user, an indication of a change in wellness of the user, an indication of a change in diagnosis, etc.).

Holistic processor 128 may receive a request to generate a holistic treatment process for a particular user. The request may include a user identifier of the particular user and an identification of one or more symptoms for which the holistic treatment process is intended to address. Holistic processor 128 may use feature extractor 132 to generate a feature vector from data from user data 136 and holistic treatment processes 140. Feature extractor 132 may extract features from the data from user data 136 and holistic treatment processes 140 and define a feature vector from the extracted features. The feature vector may organize the data according to an additional dimension such as, but not limited to, time. For example, each feature may correspond to a time instant in which the feature was originally received and/or derived. Feature extractor 132 may define a feature vector as a sequence of features over a particular time interval.

The particular time interval may be dynamically defined based on the one or more symptoms of the particular user, machine-learning models (e.g., such as an accuracy metric of the particular model, a quantity of training data used to train a particular model, a particular model to be used, an age of the particular model to be used, etc.), one or more of the holistic treatment processes received from holistic treatment processes 140, a particular type of holistic treatment process to be generated, combinations thereof, or the like. For example, if the user reports a new symptom that only recent occurred, then the particular time interval may be short (e.g., including data from when the new symptoms were reported). If the user reports an old symptom that has been occurring for a long time, then the particular time interval may be longer. A feature vector with a shorter time interval may include fewer features, but those features may be highly correlated with the new symptom. A feature vector with a longer time interval may include more features enabling the machine-learning model to generate more accurate predictions.

Holistic processor 128 may receive a request to generate a holistic process, update a user status (e.g., based on user feedback, an execution of one or more holistic treatment processes, feedback from component processors 120, and/or the like), or the like that includes information associated with the request (e.g., an identification of one or more symptoms, a time interval since each symptom was detected, an identification of a user, an identification of a holistic treatment process type to be generated, etc.). Holistic processor 128 may use request and the information associated with the request to determine the particular time interval for feature extractor 132. In some instances, holistic processor 128 may vary the time interval based on a current status of server 104 or the user to, for example, increase an accuracy of an output from machine-learning models 144, reduce resources consumed to generate an output (e.g., a quantity of memory, a quantity of available processing capacity, a quantity of data, a quantity of network bandwidth, a quantity of power needed to generate an output, etc.), reduce a time interval between receiving the request and transmitting the output, and/or the like.

Machine-learning models 144 may include one or more machine-learning models trained to provide services to user devices and/or component processors 120 such as, but not limited to, generating holistic treatment processes for users, generating predictions associated with users based on user data 136 and/or holistic treatment processes 140, generating or updating a status of the user (e.g., based on one or more interdependent holistic classes), and/or the like. In some instances, machine-learning models 144 may include one or more machine-learning models for each service provided by machine-learning models 144. In other instances, one or more services may be provided by a same model (e.g., a single machine-learning model, an ensemble model including two or more interconnected machine-learning models, etc.). Examples of such machine-learning models include, but are not limited to, perceptrons, decision trees, Naïve Base, a regression-based model (e.g., such as a logistic, etc.), neural network, deep learning networks, support vector machines (SVM), Naïve Bayes, K-nearest neighbor, random forest, combinations thereof, or the like.

The machine-learning models may be trained using training data received or derived from user data 136 and/or holistic treatment processes 140, users, component processors 120, and/or the like. In some instances, holistic processor 128 may define training thresholds based on the particular machine-learning model being trained. The training thresholds may correspond to a quantity of training data, a type of training data, and/or the like. For example, if the quantity of training data is less than a threshold quantity of training data or does not correspond to a threshold training data type, holistic processor 128 may generate and/or identify additional data that can be used to augment the training data. Holistic processor 128 may generate additional data procedurally (e.g., using semi-automated or automated software processes, etc.), manually, a combination thereof, or the like.

Alternatively, or additionally, holistic processor 128 may identify training data corresponding to one or more analogous users that may be usable to augment the training data for a user. For example, some machine-learning models may be trained for a particular user. If holistic processor 128 does not have sufficient data to generate training data to train a machine-learning model for a new user, then data known about the new user may be used to identify an analogous user. Holistic processor 1289 may identify data associated with the analogous user (e.g., such as, but not limited to, the training data used to train a machine-learning model for the analogous user, etc.) that can be used to augment the data of the new user to enable training the machine-learning model for the new user. The analogous user may be identified based one or more common attributed between the new user and the analogous user such as, but not limited to, common demographic data, common symptoms, common responses to data requests from a holistic application of server 104, social media connections, family connections, user input, component processor 120 input, combinations thereof, or the like.

Holistic processor 128 may transmit the training data for a particular machine-learning model to feature extractor 132. Feature extractor 132 may define a set of feature vectors from the training data. The set of feature vectors may be used to train the particular machine-learning model. Machine-learning models may be trained using supervised training, supervised training, semi-supervised training, reinforcement training, combinations thereof, or the like. The training phase for a particular machine-learning model may be based on a target accuracy of the machine-learning model. For example, a machine-learning model may be trained until the target accuracy is reached. In some instances, the machine-learning model may be trained until the target accuracy is reached or one or more other criteria is met (e.g., such as time, efficiency, and/or the like). For example, if a threshold time interval expires before the machine-learning model reaches the target accuracy, then the training phase may be restarted (e.g., with a new machine-learning model) or the training data may be analyzed to determine if the training data is sufficient in quantity and/or type to train the machine-learning model.

Once trained, the machine-learning models may be executed (e.g., by holistic processor 128, CPU 108, by a user device, component processors 120, etc.) to generate predictions for a user and/or user device. For example, mobile device 156 may execute a holistic application. Mobile device 156 may transmit a request for a holistic treatment process to holistic processor 128 at server 104. The request may include a user identifier of mobile device 156, an identification of one or more symptoms, and an indication of a holistic treatment process type. Holistic processor 128 may receive the request and identify data associated with the user of mobile device 156 in user data 136. Alternatively, or additionally, the data associated with the user of mobile device 156 needed to generate a holistic treatment process may be transmitted by mobile device 156 and/or one or more other devices with the request. Holistic processor 128 may identify one or more machine-learning models of machine-learning models 144 and/or an ensemble model of machine-learning models 144 based on the one or more symptoms, the holistic treatment process type, and/or the data associated with the user. Feature extractor 132 may then extract features from the data associated with the user and define a feature vector based on the identified one or more machine-learning models and/or ensemble model, the one or more symptoms, the holistic treatment process type, and/or the like. Holistic processor 128 may execute the identified one or more machine-learning models and/or ensemble model using the feature vector as input.

The machine-learning models and/or ensemble model may generate a holistic treatment process that may be particular to the user and/or mobile device 156. The holistic treatment process may be transmitted to mobile device 156. The holistic treatment process may include instructions that may be executed by the holistic processing application executed by mobile device 156. The instructions, when executed, may provide one or more processes for the user to execute. The one or more processes may be related to one or more interdependent holistic classes such that when executed by the user work together to holistically alleviate the one or more symptoms of the user. For example, the one or more processes may include a process for each of one or more interdependent holistic classes such that when executed the combined execution of the one or more processes each interdependent class provide a holistic remedy. Examples of interdependent holistic classes include, but are not limited to a treatment class, a food class, a mind class, a supplement class, and a fitness class.

In some instances, one or more of the one or more processes may be executed by one or more component processors 120. For example, a process of the one or more processes may include meditation with feedback using a meditation application (e.g., such as Calm, Aura, etc.) hosted by a component processor 120. In those instances, holistic processor 128 may transmit a portion of the holistic treatment process to a component processor 120 associated with a particular interdependent holistic class (e.g., a mind class in the previous example) along with a user identifier of the user and/or device identifier of mobile device 156, an identification of the one or more symptoms, and identification of the holistic process, data used to generate the holistic process, and/or the like. The component processor 120 may be selected based on being associated with the particular interdependent holistic class, previously selected by the user and/or previously utilized by the user, a rating of the component processor, user input, input from a component processor (e.g., such as a therapist/psychiatrist, etc.), and/or the like. The selected component processor 120 may establish a connection mobile device 156 to provide a holistic treatment process (or portion thereof) in a particular interdependent holistic class, determine a status of the holistic treatment process for the particular interdependent holistic class, determine a status of the user relative to the particular interdependent holistic class, combinations thereof, or the like.

The holistic processing application and/or selected component processors 120 may generate data associated with the holistic treatment process and/or the user during execution of the holistic treatment process and/or after termination of the holistic process. For example, mobile device 156 may receive feedback from component processors 120 and/or from the user indicating a progress of the holistic treatment process in a particular interdependent holistic class and/or overall. The feedback may be transmitted to holistic processor 128 and stored in holistic treatment processes 140 in association with the holistic treatment process generated by machine-learning models 144. In some instances, the feedback may be passed to the one or more machine-learning models and/or ensemble model that generated the holistic treatment process for reinforcement learning. In those instances, holistic processor 128 may analyze the feedback to determine the suitability of the feedback for reinforcement learning (e.g., based on content, format, a current accuracy metric of the one or more machine-learning models and/or ensemble model, etc.). If the feedback is determined to be suitable, then feature extractor 312 may extract features from the feedback that can be passed to the one or more machine-learning models and/or ensemble model for the reinforcement learning.

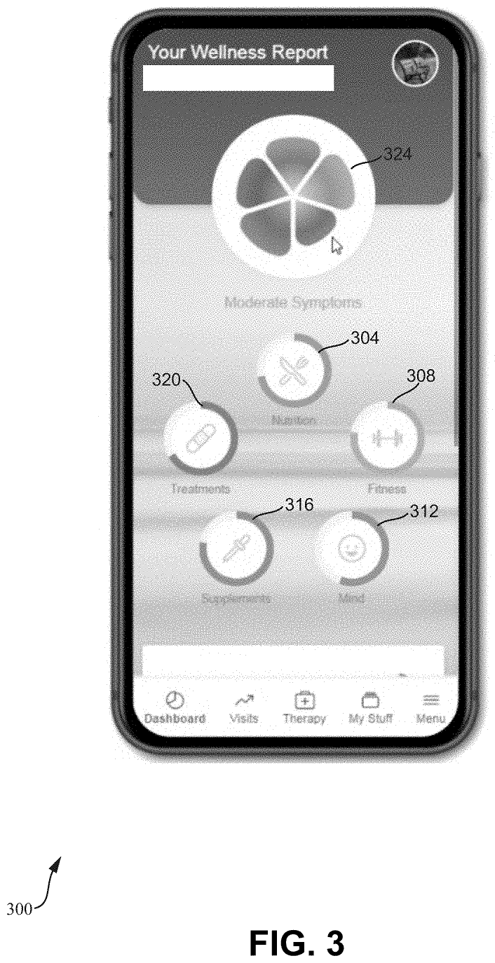

FIG. 3 illustrates an example graphical user interface of a holistic processing application depicting a status of a user according to aspects of the present disclosure. The holistic processing application may be usable to monitor a status of a user, generate holistic treatment processes to augment an overall wellness of the user and/or a particular interdependent holistic class, monitor the execution of holistic treatment processes, refine machine-learning models based on feedback derived from execution of holistic classes, receive additional information related to an overall wellness and/or particular interdependent holistic classes (e.g., articles, audio and/or video media, connections to expert systems, etc.), receive additional information about the holistic processing application and/or the holistic treatment processes generated by the holistic processing application (e.g., articles, audio and/or video media, connections to expert systems, etc.), etc.

The graphical user interface depicted includes a status of a user in five interdependent holistic classes (e.g., a treatment class, a food class, a mind class, a supplement class, and a fitness class, etc.). The graphical user interface may be configured display additional interdependent holistic classes, interdependent holistic subclasses, and/or the like. The graphical user interface may be configured to display fewer and/or different interdependent holistic classes, interdependent holistic subclasses, and/or the like. For example, a user of the holistic processing application may select one or more interdependent holistic classes to be displayed by the graphical user interface. In some instances, some interdependent holistic classes may be preselected for display by the graphical user interface (and which cannot be deselected by the user). User input may be received selecting additional interdependent holistic classes to the preselected interdependent holistic classes. Alternatively, user input may be received removing one or more of the preselected interdependent holistic classes and/or adding additional interdependent holistic classes.

Each interdependent holistic class may be associated with a value indicative of the wellness of a user in the interdependent holistic class. The value may have a minimum value and a maximum value such that the value can be represented as a percentage of the maximum value. The value may be represented graphically as a ring surrounding a symbol of the interdependent holistic class with a portion of the ring filled in with a particular color based on the value. For example, the value of interdependent holistic class 304 (e.g., the food class), is approximately 70% and the ring surrounding the symbol for interdependent holistic class 304 is filled in approximately 70%. The value may be represented by any graphical illustration such as, but not limited to, numerically (e.g., as a single value, ratio of values, percentage, etc.), as a textual description (e.g., such as a grade between A-F, a descriptive phrase, etc.), a symbol (e.g., one for each value that can be assigned to interdependent holistic class, etc.), a color (e.g., red, yellow, green, etc.), a graph, combinations thereof, or any other means to graphical convey the value to the user.

In the graphical user interface shown, interdependent holistic class 304 may represent a food class (e.g., a previously described), interdependent holistic class 308 may represent a fitness class, interdependent holistic class 312 may represent a mind class, interdependent holistic class may represent a supplement class 316, interdependent holistic class 320 may represent a treatments class.

The value may be derived based on user input (e.g., from the user, a component processor, an expert system, and/or the like), and/or one or more machine-learning model. In some examples, the holistic processing application may generate a sequence of data requests for information associated with the user and/or any symptoms reported by the user. Each request may include one or more predetermined responses for selection by the user. Alternatively, or additionally, the user may provide alphanumeric responses to one or more requests in addition to or in place of a predetermined response. The sequence of requests may be general or particular to an interdependent holistic class. The sequence of requests may be predefined and/or based on one or more decision trees. For example, an initial sequence of data requests may be defined. Upon receiving a response to a first request, the holistic processing application may determine if a follow up, related, and/or additional request should be presented before continuing to the next request in the sequence of data requests. The responses from the sequence of data requests (e.g., including responses to any follow up, related, and/or additional data requests presented) may be used to determine an overall value (e.g., if the sequence of data requests corresponds to general data requests) or a value for a particular interdependent holistic class. If the sequence of data requests corresponds to a particular interdependent holistic class, then additional sequences of data requests may be presented for other interdependent holistic classes.

The holistic processing application may define a value for each presented by the graphical user interface of FIG. 3. An overall value may include a representation of the individual values of each interdependent holistic class. For example, overall value 324 may be represented as a graphical element having five components (e.g., one for each interdependent holistic class 304-320, etc.) with the size, shape, and/or color of each component being based on the values of the corresponding interdependent holistic class. In the example graphical user interface shown, a higher value (e.g., a higher degree of wellness) in an interdependent holistic class may result in a larger component of overall value 324 and a lower value (e.g., a lower degree of wellness) in an interdependent holistic class may result in a smaller component of overall value 324.

In some instances, an overall value may be derived from the values of each interdependent holistic class. For example, an overall value may be derived by summing or averaging the values of the interdependent holistic class to generate a signal value. Alternatively, the overall value may be generated based on the values of each interdependent holistic class and a degree of dependence each interdependent holistic class may have on other interdependent holistic classes. Weights may be assigned to each interdependent holistic class based on a default degree of dependence (e.g., a weight of 1 indicating no dependence or weight based on dependences between interdependent holistic classes identify in other users such as users having similar characteristics to this user, etc.), a predicted degree of dependence (e.g., generated by a machine-learning model based user data collected by the holistic processing application, holistic treatment processes executed and/or generated for the user, similar users to this user, and/or the like), a measured degree of dependence (e.g., based on feedback from execution of holistic treatment process or treatment protocols of holistic treatment processes, etc.), combinations thereof, or the like.

For example, initial weights may be generated for each interdependent holistic class for a particular user. The initial weights may be set a value indicating no dependence between interdependent holistic classes (e.g., such as 0, 1, etc.). Alternatively, the initial weights may be derived from average weights of each interdependent holistic class relative to another interdependent holistic class from a set of users. The set of users may be selected based on characteristics in common with the particular user (e.g., such as demographic information, etc.). As the particular user provides information to the holistic processing application (e.g., demographic information, symptoms, fitness, nutrition, mental health, etc.), a profile of the particular may be generated. Features may be extracted from the profile and used as input to a machine-learning model configured to generate predicted weights for each interdependent holistic class relative to another interdependent holistic class. The predicted weights may be updated (in real-time or in a batch process) each time additional information about the user is generated and/or received (e.g., such as, but not limited execution of a holistic process, feedback after execution of a holistic treatment process, reporting new symptoms, etc.). In some instances, the predicted weights may be replaced by measured weights as the holistic treatment processing application measures the degree of dependence between interdependent holistic class for the particular user. The overall value may be generated based on the weights and the values of the interdependent holistic classes (e.g., such as by a weighted sum, etc.).

The graphical user interface may include additional information related to the user such as current symptoms reported by the user, selectable icons to search for additional information associated with a particular interdependent holistic class or holistic treatment process (e.g., holistic therapy), selectable icons to navigate to other graphical user interfaces (e.g., such as the dashboard graphical user interface shown, a visits graphical user interface with information on historical or future visits with expert systems, a therapy graphical user interface to request a holistic or medical therapy, a “my stuff” graphical user interface to display information associated with the user and the holistic processing application, a menu graphical user interface with additional selectable icons, etc. The information associated with the user and the holistic processing application (e.g., in the “my stuff” graphical user interface) can, but not limited to, user information collected by the holistic processing application, previously generated and/or holistic treatment processes, previously reported symptoms, symptoms previously indicated as alleviated, an identification of previous searches performed through the holistic processing application, etc.

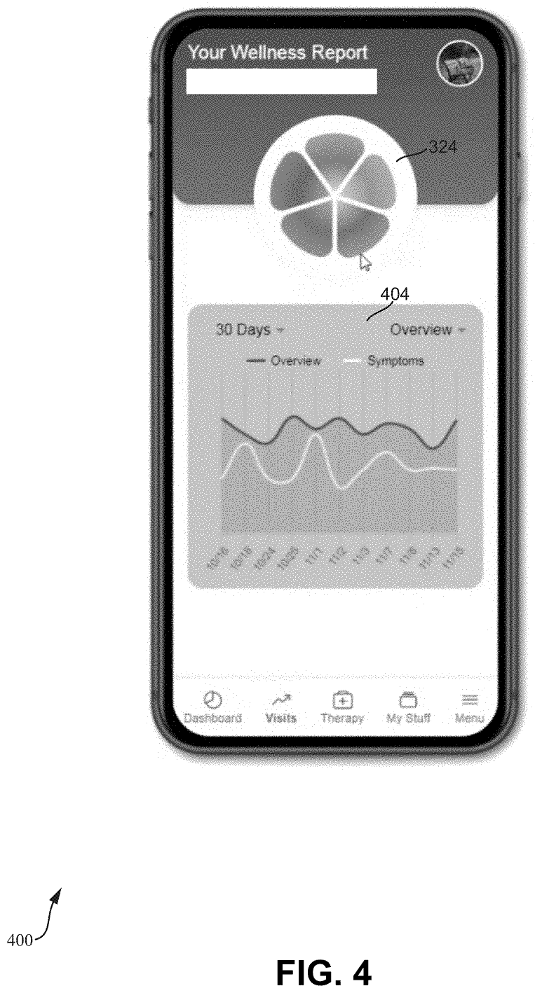

FIG. 4 illustrates an example graphical user interface 400 of a holistic processing application depicting a status of a particular interdependent holistic class according to aspects of the present disclosure. The holistic processing application may present selectable graphical elements that may provide additional information. For example, each of interdependent holistic classes 304-320 and overall value 324 of the graphical user interface of FIG. 3 may be selected to provide a new graphical user interface with additional information. In some instances, the orientation, format, shape, size, and/or color of the new graphical user interface may be based on historical user interaction with the holistic processing application and/or the previous graphical user interface from which the user may navigate to the new graphical user interface.

If user input selecting overall value 324 is selected, then holistic processing application may provide the example graphical user interface 300 of FIG. 3, which may provide additional information about overall value 324. For example, graphical element 404 includes a representation of an example additional information that may be displayed. Graphical element 404 includes a graphical representation of the historical values of overall value 324 over a previous time interval. In some instances, other data may also be included in the same graphical representation such as symptoms (as shown), the value of one or more interdependent holistic classes, etc. The previous time interval may be set to predetermined default setting (e.g., 1 day, 30 days as shown, etc.). The predetermined default setting may be modified by user input selecting a particular time interval. Once selected the graphical element 404 may change to include the additional information over the selected time interval. In some instances, the previous time interval may be dynamically selected based on a machine-learning model, historical previous time intervals selected by the user, etc.

Example graphical user interface 400 may include other forms of additional information and/or graphical elements. For example, if user input is received selecting an interdependent holistic class (e.g., from example graphical user interface 300 or from graphical user interface 400), then graphical element 404 be modified to include the values of the selected interdependent holistic class over the previous time interval. Example graphical user interface 300 may include other additional information such as, but not limited to, demographic data, user data, symptom data, feedback data, an identification of previous holistic treatment processes executed by the user (e.g., alone or correlated with overall value 324, a value of an interdependent holistic class, symptom data, or other data), a progress of currently executing holistic treatment processes, combinations thereof, or the like.

The form of any content presented by a graphical user interface of the holistic processing application may include, but is not limited to, graphics, icons, graphs (e.g., as shown by graphical element 404), alphanumeric text, audio, and/or video). For example, the holistic processing application may provide an audible presentation of any or all of the data presented by example graphical user interface 400.

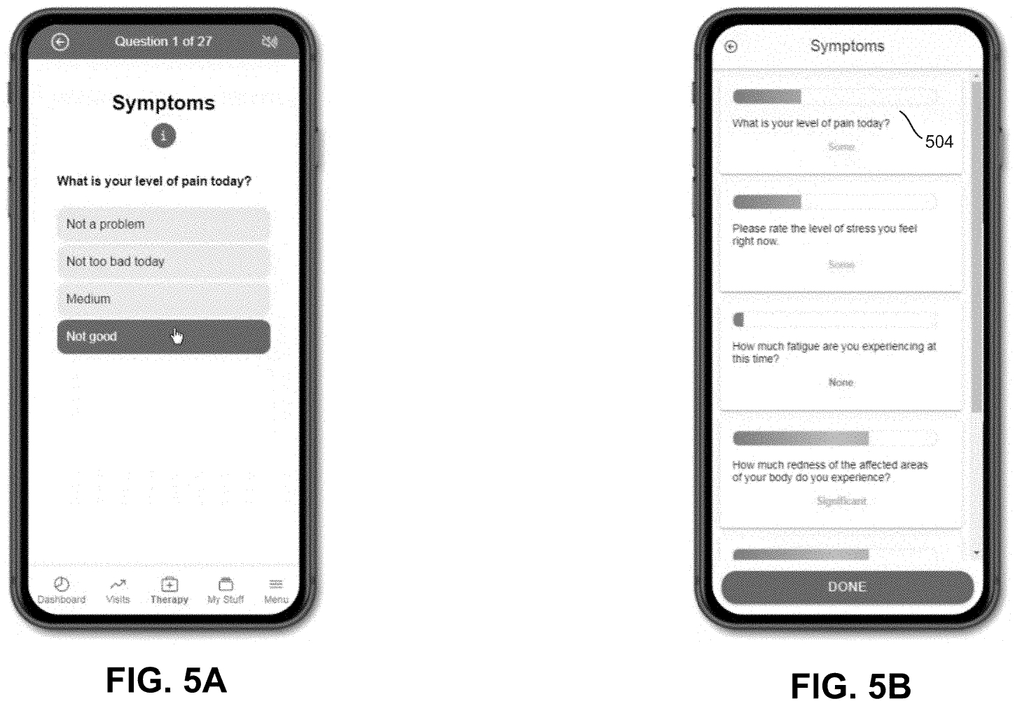

FIGS. 5A and 5B illustrate example graphical user interfaces of a holistic processing application depicting acquisition of information usable to define holistic treatment process and/or a status of a user according to aspects of the present disclosure. A holistic processing application may first gather user data to generate an initial status of a user. The holistic processing application may request access to data that may already be stored on the computing device on which the holistic processing application is executing. The holistic processing application may include one or more interfaces (e.g., application programming interface, user interface, etc.) that enable the holistic processing application to access native systems of the computing device (e.g., such local or remote storage, operating system data, etc.) and/or convert data in a native format of the computing device into a format of the holistic processing application. Through the interfaces the holistic processing application may gather the user data stored by the computing device. In some instances, the holistic processing application may include interfaces that enable communications with externally connected devices (e.g., devices connected via Bluetooth, Wi-Fi, etc.). For example, the computing device may be connected to a smartwatch via Bluetooth. The holistic processing application may include interfaces that can obtain sensor data captured by the smartwatch.

The holistic processing application may also request information from the user by presenting a sequence of data requests to the user as shown in FIG. 5A. The sequence of data requests may be statically defined (e.g., the same sequence of data requests may be presented to all new users) or may be dynamically defined based on information already known about the user and/or responses received from previous requests of the sequence of data requests. For example, a first request may request symptom information such as a current pain level of the user. The holistic processing application may select and present a subsequent request based on the response received to the first request such as requesting information associated with a type or location of the reported pain. In some instances, a sequence of data requests may be defined for a user in regular intervals to generate a historical profile of the user. The regular intervals may be defined to maximize a frequency of information received from the user while minimizing obtrusiveness. In some examples, a sequence of data requests may be presented to the user once a week, once every two weeks, once a month, etc. The holistic processing application may define the regular interval based on user input, characteristics received and/or generated about the user, an indication of a degree of user satisfaction with the frequency with which the sequence of data requests is presented, combinations thereof, or the like. In other examples, the regular interval may be based on characteristics of the user and/or holistic processing application such as, but not limited to, an occurrence of an event, a request to generate a holistic process, one or more constraints being satisfied, and/or the like.

The holistic processing application may receive a response to a request via same graphical user interface that presents the request (as shown in FIG. 5A) or via a subsequent graphical user interface. User input may include one or more preset responses, alphanumeric text, a slider bar or other means to provide numerical input, audio, and/or video. Natural language input received via alphanumeric text, audio, and/or video may be parsed by a sequence of machine-learning models. A first machine-learning model may be configured to convert audio to text (e.g., so called speech-to-text models), a second machine-learning model may be configured to parse the natural language text into a recognizable response by the holistic processing application. A third machine-learning model may process video of the user to identify additional contextual data in a response (e.g., gestures, body language, etc.), that may add to or augment the recognizable response generated by the second machine-learning model. The first and second machine-learning models may be recurrent neural networks (e.g., such as, but not limited to, long short-term memory, gated neural network, etc.).

The third machine-learning model may be convolutional neural network, a recurrent neural network, or an ensemble model that includes both a convolutional neural network and a recurrent neural network. The convolutional neural network may execute using features derived from one or more frames of the video and perform image classification (e.g., indicating a probability in which a frame or a portion thereof includes a particular gesture, body language, etc.). In some examples, the convolutional neural network may identify portions of each frame and indicate a probability that the portion of the frame include a particular gesture and/or body language. Features may be derived from each portion of each frame and the probabilities and passed as input into the recurrent neural network. The recurrent neural network may then predict a new probability that a gesture or body language is present based on the portion of the frame input (and the portion of one or more previous frames due to the recurrent neural network preserving data from previous inputs). In some instances, a classifier may be used to predict a context associated with a particular gesture or body language. The classifier may receive the natural language text and/or the recognizable response and the new probability. Alternatively, the recurrent neural network may include the classifier (e.g., as an output layer of the neural network).

Upon receiving responses to the sequence of data requests, the holistic processing application may generate a summary graphical user interface (e.g., as shown in FIG. 5B). The summary graphical user interface may include a representation of a response to each request such as a graphic indicating a degree of a response relative to a minimum value and/or maximum value for that response. The graphic may be displayed with a representation of the request to indicate a context of the response. As shown, the response to the first request (“Not Good” in FIG. 5A) may result in graphic 504. A categorical value may be included or associated with graphic 504. The categorical value “some” is associated with graphic 504. In some alternative examples, the categorical value may be replaced with a numerical value indicative of the degree.

In some instances, the second sequence of data requests may be presented to the user at a later time (e.g., based on the regular time interval, etc.). The summary graphical user interface may be augmented to include the responses to the second sequence of data requests. In some instances, if a request is included in both sequences, then the graphic presented within the summary graphical user interface may represent the newer response (e.g., the response to the request in the second sequence of data requests). In other instances, the graphic presented within the summary graphical user interface may be based on both the response from the first sequence of data requests and the response from the second sequence of data requests. For example, the graphic may represent a summation of both responses, a weighted sum of both responses (with the newer response being weighted higher than older responses), an average, median, and/or the like.

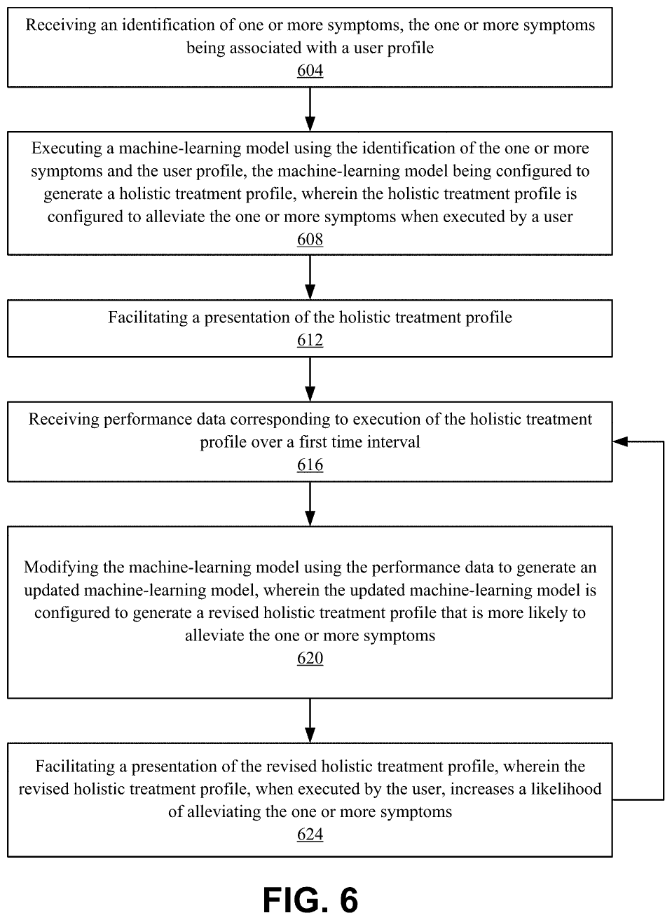

FIG. 6 illustrates a flowchart of an example process for generating holistic treatment processes executable to improve an overall wellness of a particular user according to aspects of the present disclosure. At block 604, a computing device receives an identification of one or more symptoms, the one or more symptoms being associated with a user identifier. The computing device may be a device operated by a user and executing a holistic processing application such as, but not limited to, a desktop or laptop computer, tablet, mobile device, and/or the like. Alternatively, the computing device may be a device hosting a holistic processing application configured for execution by one or more connected devices. The computing device may identify user data (e.g., such as user data stored in user data 136 of FIG. 1) that corresponds to the user identifier and associate the user data with the one or more symptoms. The user data may include information associated with a user of the computing device and/or a holistic processing application such as, but not limited to, demographic information, previously reported symptoms, ahistorical values for one or more interdependent holistic classes, a historical overall value, responses to sequences of data requests, results derived from responses to sequences of data requests (e.g., such as those depicted by summary graphical user interface of FIG. 5B), an identification historical holistic treatment processes executed by the user, a status of a current holistic treatment process executed by the user, feedback from the user, feedback from one or more component processors, feedback from expert systems, historical and/or current sensor data associated with the user (e.g., such as, but not limited to, heart rate data, heart rate variability data, accelerometer data, speedometer data, electroencephalogram data, electrocardiogram data, any other data received from or associated with the user, combinations thereof, or the like.

At block 608, the computing devices executes a machine-learning model using the identification of the one or more symptoms and the user identifier. The machine-learning model is configured to generate a holistic treatment process that may alleviate the one or more symptoms when executed by a user. The holistic treatment process can include treatment protocols for a set of interdependent holistic classes, where treatment protocol may be executable by the computing device, one or more user devices and/or devices connected to the one or more user devices, by the user, by one or more component processors, by one or more expert systems, and/or the like. The set of interdependent holistic classes may include a treatment class, a food class, a mind class, a supplement class, and a fitness class. The holistic treatment process may include one or more treatment protocol for each interdependent holistic class or for one or more interdepend holistic classes. For example, a holistic treatment process may include a first treatment protocol for fitness establishing an exercise routine to be performed by the user, a second treatment protocol of a nutrition to be established by the user, a third treatment protocol establish a connection with a component processor implementing meditation-based services, etc.

The holistic treatment process may be associated with a time interval over which each treatment protocol is to be executed. The time interval may be statically defined (e.g., one week, two weeks, 1 month, etc.) or dynamically defined. The time interval may be established by the machine-learning model based on the one or more symptoms and/or the user data associated with the user identifier. For example, if previous holistic treatment processes executed unsuccessfully or did not alleviate the symptoms, then the machine-learning model may increase the time interval to increase a probability that the holistic treatment process will alleviate the symptoms. Alternatively, or additionally, user input may be received selecting or contributing to the selection (e.g., with the machine-learning model output, or other data) of the time interval.

At block 612, the computing device facilitates a presentation of the holistic treatment process. In some instances, facilitating the presentation of the holistic treatment may include displaying the holistic treatment process and/or details thereof in a graphical user interface. The computing device may cause a holistic processing application to generate a graphical user interface that includes a presentation of the holistic treatment process. The graphical user interface may then be displayed by the holistic processing application. In other instances, facilitating the presentation of the holistic treatment process may include transmitting the holistic treatment process and/or details thereof to a holistic processing application executing on a remote device (e.g., such as a mobile device, desktop or laptop computer, etc.). The holistic treatment process may include treatment protocols to be executed by a component processor, expert system, and/or the like. For example, as shown in FIG. 2, a treatment protocol may include a meditation application hosted by remote device 166 and/or service provider. The treatment protocol may facilitate a connection between the computing device (or the device executing the holistic processing application such as user device 156) and the remote device 166 and/or service provider through network 124 to enable access to the meditation application. The meditation application, remote device, and/or service provider may monitor interactions with the computing device (or the device executing the holistic processing application) and provide information associated with those interactions to the holistic processing application and/or the computing device.

The presentation of the holistic treatment process may enable the user to review details of the holistic treatment process, review treatment protocols for each interdependent holistic class, review the predicted degree of interdependence between each interdependent holistic class (e.g., as output from the machine-learning model, etc.), identify component processors that are to facilitate execution of one or more treatment protocols, identify one or more expert systems that are to facilitate execution of one or more treatment protocols, modify the holistic treatment process, modify one or more treatment protocols, provide feedback regarding the holistic treatment process and/or a current execution thereof, provide feedback regarding one or more treatment protocols and/or a current execution thereof, and/or the like.

At block 616, the computing device may receive performance data corresponding to execution of the holistic treatment process over a first time interval. Executing the holistic treatment process may include execution of each treatment protocol of the holistic treatment process. In some instances, the treatment protocols can be executed in parallel (e.g., as approximately a same time). The treatment protocols may execute at varying rates (e.g., once a day, multiple times a day, once a week, once over the first time interval, etc.), which may cause some treatment protocols to terminate prior to the expiration of the first time interval. The holistic processing application may schedule execution of the treatment protocols to ensure that each treatment protocol terminates prior to or with the expiration of the first time interval. The schedule may be presented via a graphical user interface of the holistic processing application (e.g., such as within a calendar graphical user interface, etc.). Alternatively, or additionally, the schedule may be presented via push notifications and/or the like. For example, when a portion of a treatment protocol is to be executed at a particular time or there is a scheduled appointment with a component processor etc., the holistic processing application may cause a push notification to remind the user. The push notification may include an identification of the portion of the treatment protocol or appointment, a timestamp indicating a start of the portion of the treatment protocol or appointment, and/or the like.

The holistic processing application may monitor execution of each treatment protocol to generate the performance data. The holistic processing application may receive feedback from the user regarding execution of a treatment protocol. In some instances, the feedback may include the selection of one or more predetermined values corresponding a user-perceived status of a treatment protocol and/or the holistic treatment process. In other instances, the feedback may include natural language text, audio, and/or video. In those instances, the feedback may be processed by one or more of the aforementioned machine-learning models to convert natural language speech into natural language text, natural language text into parsable text, and/or gestures (e.g., or body language) within video into parsable text, etc. as previously described. The holistic processing application may receive execution information from component processors that are configured to facilitate execution of treatment protocols. The information may be received directly from a component processor or from the device executing the holistic processing application (which may receive the information directly from the component processor).

The holistic processing application may also generate execution information from sensors in communication with the holistic processing application. The sensor may include sensors of the device executing the holistic processing application and/or sensors of peripheral devices in communication with such a device. The sensors may include, but are not limited to, heart rate sensors, heart rate variability sensors, accelerometer sensors, speedometer sensors, electroencephalogram sensors, electrocardiogram sensors, and the like. For example, the peripheral device may be a smartwatch in communication with the device that executes the holistic processing application (e.g., via Bluetooth, Wi-Fi, etc.). The smartwatch may capture heart rate and/or heart rate variability, exercise patterns, sleep patterns, etc. that may be used to determine a performance of one or more treatment protocols of a corresponding one or more interdependent holistic classes.

The feedback, execution information from component processors, and the sensor-derived execution information may be used to generate the performance data. The performance data may indicate whether the holistic treatment process and/or the treatment protocol of a particular interdependent holistic class are executing as intended. The performance data may also indicate how a treatment protocol of an interdependent holistic class affects other interdependent holistic classes and/or a treatment protocols of the other interdependent holistic classes. The performance data may be used to update the values of the interdependent holistic classes and/or the overall value, adjust the weights assigned to each interdependent holistic class, modify how treatment protocols and/or holistic treatment protocols will be generated for the user in the future, modify the machine-learning model that generated the holistic treatment protocols, combinations thereof, or the like.

At block 620, the computing device may modify the machine-learning model using the performance data to generate an updated machine-learning model. The updated machine-learning model may be configured to generate revised holistic treatment processes that may be more likely than the initial holistic treatment process to alleviate the one or more symptoms. The revised holistic treatment processes may include revised treatment protocols for one or more interdependent holistic classes. In some instances, the machine-learning model may be modified before expiration of the first time interval to generate a revised holistic treatment process. The initial holistic treatment process may be terminated once the revised holistic treatment process is generated and the revised holistic treatment process may be scheduled for execution for the remainder of the first time interval.

In other instances, the revised holistic treatment process may be generated during or after the first time interval. For example, in response to an indication that the one or more symptoms have persist or have returned, the machine-learning model may generate the revised holistic treatment process, which may have a higher likelihood of alleviating the one or more symptoms than the initial holistic treatment process. The revised holistic treatment process may be configured for execution over a second time interval that may either overlap with but extend beyond the first time interval or begin after the first time interval. The second time interval may be determined by the updated machine-learning model to increase a likelihood that the revised holistic treatment process will alleviate the one or more symptoms.

Modifying the machine-learning model may include having the machine-learning model generate revised weights for the interdependent holistic classes. In some instances, the machine-learning model may be modified by generating feature vectors from the performance data and training the machine-learning model using the feature vectors. In other instances, the machine-learning model may be modified by adjusting internal node weights of the machine-learning model using the performance data. In those instances, the machine-learning model may be modified automatically (e.g., by processing the performance data with the machine-learning model and/or another machine-learning model) or manually. The performance data may be indicative of a degree of dependence that a particular interdependent holistic class has on one or more other interdependent holistic classes for that user. The performance data may be usable by the machine-learning model and/or by a user to generate revised weights for each interdependent holistic class. The holistic processing application may replace current weights with the revised weights based on the performance data.

The updated machine-learning model may incorporate the revised weights to generate treatment protocols that take advantage of the degree of dependence between interdependent holistic classes. For example, the updated machine-learning model may be configured to generate revised holistic treatment processes for the user by adjusting a treatment protocol associated with a first interdependent holistic class of the set of interdependent holistic classes and one or more additional interdependent holistic classes that depend on the first interdependent holistic class.

In one illustrative example, the performance data may indicate that the fitness class has a higher degree of dependence on a nutrition class for a particular user. The machine-learning model may generate revised treatment protocols for both the fitness class and the nutrition class in the next holistic treatment process. The revised nutrition treatment protocol may have an increased effect on both the treatment protocol for the fitness class (e.g., increase the likelihood that the fitness treatment protocol will terminate successfully) and the fitness class (e.g., increase a value improvement of the fitness class from execution of the revised nutrition treatment protocol and the revised fitness treatment protocol, increase the likelihood of the revised treatment protocols contributing with other treatment protocols to alleviate the one or more symptoms, etc.).

At block 624, the computing device facilitates a presentation of the revised holistic treatment process. In some instances, facilitating the presentation of the revised holistic treatment may include displaying the holistic treatment process and/or details thereof in a graphical user interface of the holistic processing application, another application, or via another medium (e.g., text messaging, direct messaging, email, etc.), etc.). In other instances, facilitating the presentation of the holistic treatment process may include transmitting the holistic treatment process and/or details thereof to a holistic processing application executing on a remote device (e.g., such as a mobile device, desktop or laptop computer, etc.). The computing devices may facilitate the presentation of the revised holistic treatment process in a same manner as described by block 612 or in a different manner.

The presentation of the revised holistic treatment process may enable the user to review details of the revised holistic treatment process, review the treatment protocols for each interdependent holistic class in the revised holistic treatment process, review any revisions included to the revised holistic treatment process from the initial holistic treatment process, review any revisions to the treatment protocols included in the revised holistic treatment process from the initial holistic treatment process, review the predicted degree of dependence between each interdependent holistic class (e.g., weights output from the machine-learning model, user input etc.), review the measured degree of dependence between each interdependent holistic class based on the performance data (e.g., revised weights output from the machine-learning model, user input, and/or the like), review and/or identify component processors that are to facilitate execution of one or more treatment protocols of the revised holistic treatment process, identify one or more expert systems that are to facilitate execution of one or more treatment protocols of the revised, modify the holistic treatment process, modify one or more treatment protocols of the revised, provide feedback regarding the revised holistic treatment process and/or a current execution thereof, provide feedback regarding one or more treatment protocols of the revised holistic treatment process and/or a current execution thereof, and/or the like.

The presentation may also enable the user to execute the revised holistic treatment process, transmit the revised treatment process (e.g., to devices corresponding to one or more users associated with the user, a medical professional, etc.), broadcast the revised treatment process (e.g., via a social media post, etc.), and/or the like. Executing the revised holistic treatment process may improve a likelihood of alleviating the one or more symptoms (over the original holistic treatment process, etc.).

In some implementations, the operations, format, and/or interfaces (e.g., graphical user interfaces, etc.) of the holistic processing application may be dynamically defined (e.g., at runtime) based on instructions received from a server (e.g., such as server 104) and/or one or more other remote devices (e.g., devices executing holistic processing applications operated by other users, servers, databases, etc.). In those instances, the holistic processing application may operate as a distributed service which may include one or more processes executing on a computing device operated by a user and one or more processes executing on a server. Alternatively, the holistic processing application may be a standalone application that receives executable instructions in real-time that provides some or all of the functionality of the holistic processing application. When operating as distributed service, the processes executed by computing devices operated by users may be semi-isolated to reduce or prevent data associated with the user from being transmitted between computing devices.

In some examples, the holistic processing application may include instructions that when executed by a computing device operated by a user may provide basic functionality of the holistic processing application. For example, the basic functionality may include instructions that enable the holistic processing application to execute and provide a limited set of operations such as, but not limited to providing particular interfaces (e.g., such as splash interfaces, loading interfaces, login interfaces, etc.), provide general information associated with the holistic processing application, input/output processing, and/or the like. The basic functionality may be adjusted based on the device executing the holistic processing application. For example, the basic functionality included in a holistic processing application executed by a mobile device may include a first set of functions and the basic functionality included in a holistic processing application executed by a computing device may include a larger, second set of functions due to the additional processing resources available to computing devices.

In some instances, the basic functionality may be user agnostic such that each holistic processing application executing on a same computing device type may include the same basic operations. By maintaining the basic functionality, the holistic processing application may avoid retaining sensitive information associated with the user which may be accessible by other users of the computing device, other process of the computing device or other users of the computing device. Upon executing the holistic processing application, access credentials may be provided (e.g., by the computing device, by the holistic processing application, using a token service, by the user, etc.) to authenticate the particular computing device and/or the user executing the holistic processing application. Alternatively, an altered version of the access credentials may be provided, such as, but not limited to, a hash derived from the access credentials, a salted hash derived from the access credentials, etc.). A process (or another instance) of the holistic processing application executing on a server may then authenticate the computing device and/or the user using the access credentials and transmit additional instructions and/or information to the holistic processing application executing on the computing device to enable additional functionality of the holistic processing application.

The additional functionality may include functions that are user agnostic (e.g., instructions, application updates, etc.) and/or functions that are user specific (e.g., the current holistic treatment process, treatment protocols, functions associated with a previous execution of the holistic processing application, etc.). The additional information may include data that is user agnostic (e.g., news, application updates, a last state of the holistic processing application when it terminated, application information, instructions, etc.) and/or data that are user specific (e.g., information associated with current or previous holistic treatment processes, information associated with current or previous treatment protocols, personal identifiable information, user profile information, information associated with contacts of the user, values associated holistic treatment classes, information associated with communications transmitted and/or received by the user, health information of the user, medical professionals connected to or providing care to the user, information stored or generated during a previous execution of the holistic processing application etc.).

For example, the holistic processing application may receive information associated with the user and/or a previous execution of the holistic processing application, may be received from an input/output interface (e.g., from the user or an entity associated with the user, etc.), one or more connected devices (e.g., such as mobile devices, sensors, wearable devices, etc.), a server (e.g., operating the instance of the holistic processing application that manages the holistic processing application operated by users, etc.), component processors, and/or the like. Some information, such as user information, may be sensitive information associated with the user such as, but not necessarily limited to, personal identifiable information (PII), health information (e.g., such as diagnoses from medical professionals or self-identified by the user, symptoms, information associated with treatment protocols, progress executing treatment protocols, etc.), personal notes, an identification of family members and/or friends, an identification of other users associated with the user, social media information, and/or the like. The additional functionality and/or information may be stored separately from the holistic processing application (e.g., in a separate address space) so as to control the additional information and avoid corrupting the basic functionality or other instructions useable to execute the holistic processing application.

The holistic processing application may receive the additional functions and/or information dynamically as the holistic process application executes. For example, each interface may include a layout (indicating the location of controls, text, graphics, etc.), an indication of an expected appearance (e.g., color, graphics, background, etc.), one or more controls (e.g., that can be selected to navigate to another interface, open or close interfaces, terminate the application, execute functions, request additional functions or information, transmit or receive communications, establish bi-directional communications such as a phone call or instant messaging session, etc.), and/or the like. In some examples, the layout and basic functions of the interface may be stored in local memory of the holistic processing application. Upon receiving the input, the holistic processing application may obtain additional functions and/or information to modify the interface. The modified interface may include additional or modified information, additional or modified controls (e.g., selectable object configured to perform an action upon selection, etc.), modified appearance (e.g., different colors, shading, images, graphics, backgrounds, etc.), and/or the like. The holistic processing application can dynamically (e.g., a runtime and real time) obtain the instructions configured to provide the functionality and look and feel of the holistic processing application as the user operates the holistic processing application.

When an interface is requested, the holistic processing application may determine if some or all of the data of the interface is stored in local memory. If so, the holistic processing application will load the interface or the portion thereof that using the data stored in local memory. The holistic processing application may then execute a call to the process (or instance) of the holistic process application executing on the server to obtain the data the remaining data of the interface (or the entire data of the interface if no portion was stored in local memory). In some instances, the call can include an identification the requested resource, an identification of the interface, an identification of objects of the interface, an identification of information to be presented by the interface, an identification the user and/or computing device requesting the resource, and/or the like. The process (or instance) of the holistic process application executing on the server may identify the requested resources and transmit the resources back to the computing device. The call may be executed, and the resource may be received, during the time interval between receiving the request for the interface and loading the data of the interface that is stored in local memory such that the user is unaware that the data was received from a remote source. The application may appear to execute as if all of the resources of the application are stored locally.

The holistic processing application may receive input configured to modify a portion of a particular interface. The input may identify the aspect of the particular interface that is to be modified (e.g., the layout, one or more colors, background, images and/or graphics, content, etc.) and the modification that is to be made. The holistic processing application may transmit a communication to the process (or instance) of the holistic process application executing on the server including the identified aspect and the modification. The process (or instance) of the holistic process application executing on the server may then transmit instructions to the holistic processing application that modify the particular interface according to the input. The holistic processing application may also modify one or more other interfaces based on the modification to the particular interface. The instructions may be stored within memory of the computing device and/or the server. The next time the holistic processing application is loads the particular interface (e.g., during the current execution of the holistic processing application or a subsequent execution of the holistic processing application), the modification version of the articular interface can be loaded.

The holistic process application executing on the server may be operated to modify interfaces of holistic processing applications executed by computing devices. The holistic process application executing on the server may receive input identifying a particular holistic processing application to modify (e.g., operated by a particular user, operated by class of users, executed by a particular device type, executed by a particular device, etc.), the aspect of one or more interfaces to modify, and the modifications that are to be made. The holistic process application executing on the server may then define instructions to modify the one or more interfaces according to the input such that when a targeted holistic processing application (e.g., operated by a targeted user or device) requests an interface from the holistic process application executing on the server, the holistic process application executing on the server transmits the modified version of the interface (or instructions to modify the interface into the modified version of the interface) to the targeted holistic process application. Since each interface may be loaded dynamically from instructions received from the holistic process application executing on the server in real-time, each holistic process application can be individually modified at runtime (e.g., while the application is executing and without requiring the application to be reloaded).

In an illustrative example, a user of a holistic processing application may generate a first holistic treatment process that includes a first treatment protocol defining an exercise routine and a second treatment protocol defining a diet including particular foods. The user may modify the second treatment protocol using the interface through which the second treatment protocol is presented. The modification can be an adjustment to the treatment protocol, deletion of the treatment protocol, a new treatment protocol, etc. The interface may then be modified by implementing the modification to the second treatment protocol. The modification may also cause: the process (or instance) of the holistic process application executing on the server to record the modification such that the next time the user loads the interface the modification will be maintained, the process (or instance) of the holistic process application executing on the server to modify the holistic treatment process by adjusting the other treatment protocols and/or defining new treatment protocols based on the modification, future holistic treatment processes to be generated accounting for the modification, etc. Users may modify interfaces to modify or remove interdependent holistic classes, treatment protocols, diseases, symptoms, holistic treatment processes, etc. in addition to the layout, color, graphics, background, etc.

During execution of the holistic processing application, the holistic processing application may identify and track user-specific information that may be designated as sensitive information (e.g., using a trained machine-learning model, user input, an identification of information historically designated as sensitive information, an identification of information designated as sensitive information by other users, etc.). In some examples, each discrete portion of information may include a retention flag that can be defined by the training machine-learning model, user input, etc. For information designated as sensitive information and other information designated as not to be retained, the retention flag may be set to false. For other information that is to be retained between executions of the holistic processing application, the retention flag may be set to true.

The holistic processing application may retain information until termination at which time, the holistic processing application may transmit the user-specific information usable for a subsequent execution of the holistic processing application to the process (or instance) of the holistic process application executing on the server. For example, the holistic processing application may transmit any information with a flag set to false, a current state of the holistic processing application, other data received and/or generated during execution of the holistic processing application, etc. Alternatively, or additionally, the holistic processing application may transmit information designated by the user (which may be the same as or different from the information with a flag set to false) to the process (or instance) of the holistic process application executing on the server. The holistic processing application may receive a communication confirm receipt of the transmitted information (e.g., such as a checksum, hash of the data transmitted, etc.). The holistic processing application may then delete any information with a flag set to false. The information transmitted to the process (or instance) of the holistic process application executing on the server may be downloaded the next time the holistic processing application is executed on the computing device.

In some examples, the holistic processing application may also periodically (e.g., in regular time intervals, upon detecting an event such as user selection of an interface element or a change in data stored in memory, etc.) transmit non-user state information (in addition to or in place of the user-specific information) to the process (or instance) of the holistic process application executing on the server. The non-user state information may include (e.g., a state of the holistic processing application executing on the computing device, functions executed or accessed since the holistic process application was executed, metadata, changes in data stored in memory that are not associated with the user, etc.). Alternatively, instead of transmitting the information periodically, the holistic processing application may transmit the information upon receiving a request to terminate the holistic processing application. The non-user state information may be deleted upon termination of the holistic processing application executing on the computing device (e.g., to reduce a memory footprint of the holistic processing application, etc.). By reducing the quantity of retained data, the holistic processing application can reduce the memory footprint of the holistic processing application regardless of the quantity of users that accessing the holistic processing application.

In some other instances, the basic functionality may include some user-specific functions and/or information retained from a previous execution of the holistic processing application. In those instances, the user-specific information may be stored in local memory of the computing device. By maintain the user-specific information in local memory, the process (or instance) of the holistic process application executing on the server may store little if any sensitive information associated with the user of the holistic processing application executing on the computing device, which may minimize a likelihood that the user-specific information may be improperly managed or accessed by a malicious actor.

When the holistic processing application is executed by the computing device, a first interface may be presented to request access credentials from the user. Alternatively, the first interface may obtain the access credentials from the computing device on which the holistic process application executes (e.g., from previously provided access credentials, a token, an analysis of the computing device and/or any data thereon that can provide an identification of the user, and/or the like). The holistic processing application may transmit a communication to the process (or instance) of the holistic processing application executing on the server that includes the access credentials. In some instances, the process (or instance) of the holistic processing application executing on the server may use other information in addition to or in place of the access credentials to authenticate the user of the holistic processing application executed by the computing device. For example, the holistic processing application may include in the communication: an identifier associated with the user, an identification of the device that transmitted the notification (e.g., such as, but limited to, globally unique device identifier, an identification of a software version of the holistic processing application executing on the device, a device fingerprint generated from an identification of software stored on the device and/or hardware installed on the device, combinations thereof, or the like), combinations thereof, or the like.

The process (or instance) of the holistic processing application executing on the server may authenticate the user based on the communication and establish a new session for the holistic processing application executed by the computing device. The holistic processing application may identify information and/or functions to be transmitted to the computing device for that user such as information associated with previous executions of the holistic processing application, information associated with or received from the user (e.g., such as information with a flag set to false, information received from other devices or component processors, information derived from previous executions of the holistic processing application, etc.), a state of the holistic processing application when the holistic processing application was previously executed, etc.

The holistic processing application may the transmit the identified information and/or functions to the computing device. Upon being received, the instance of the holistic processing application executing on the computing device may operate with additional functionality, additional and/or updated interfaces, etc. Since the holistic processing application may store data remotely and be dynamically augmented with functionality at runtime, the memory footprint of the portion of the holistic processing application executing on user devices can be reduced.

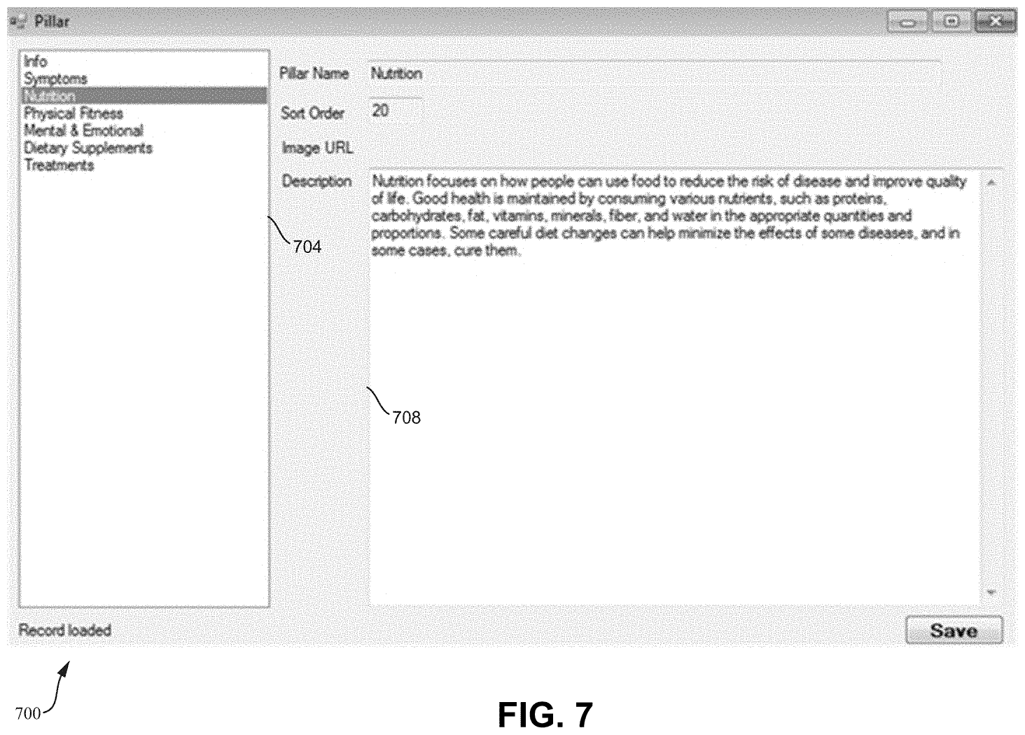

FIG. 7 illustrates an example graphical user interface of an application configured to remotely modify interfaces of a holistic processing application associated with interdependent holistic classes according to aspects of the present disclosure. The process (or instance) of the holistic processing application executing on the server may include graphical user interface 700. Graphical user interface 700 can be operated to dynamically adjust information associated with interdependent holistic classes that can be provided to the holistic processing application executing on computing devices. For example, upon selection of an interdependent holistic class in box 704, graphical user interface 700 may provide additional information associated with the selected interdependent holistic class. The information can be modified (e.g., such as through box 708) by adding and/or removing information. Upon saving the modification, the information may be approximately immediately accessible to each holistic processing application.

For example, when an interface of the holistic processing application configured to present the modified information of the interdependent holistic class is loaded, the holistic processing application may dynamically obtain the modified information from the remote device. Alternatively, the holistic processing application may obtain a delta structure that corresponds the difference between the unmodified information and the modified information. The delta information may have a reduced memory footprint and provide only enough information to convert the unmodified information to the modified information. Changes to information, interface, functions can be implemented in real-time without requiring the holistic processing application to be terminated or reloaded.

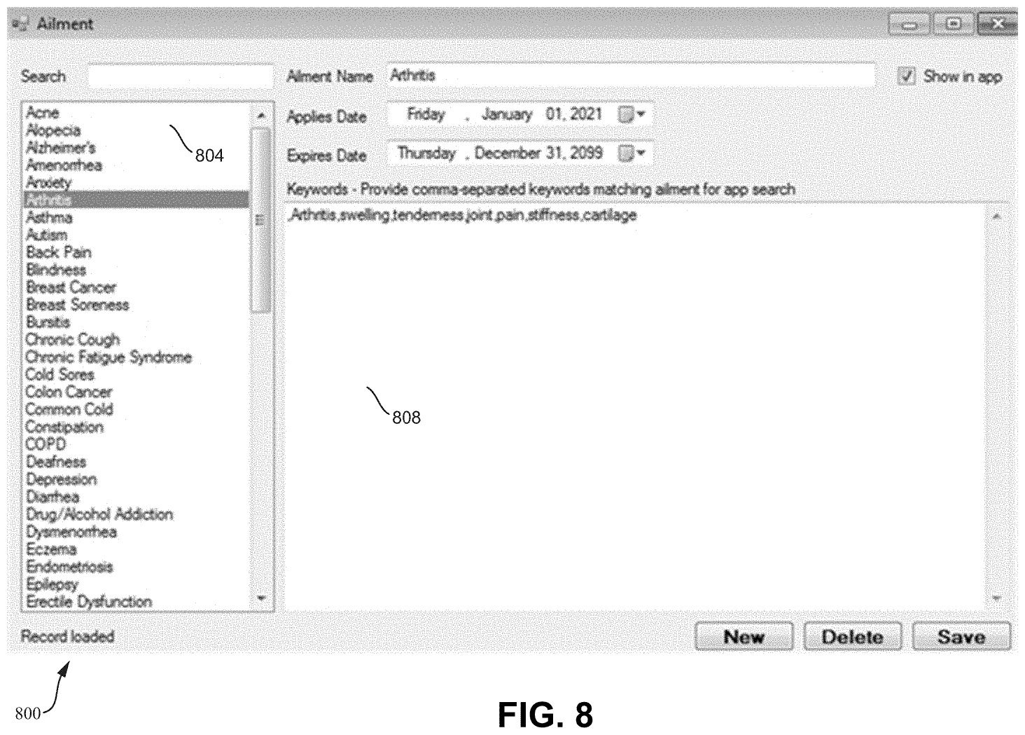

FIG. 8 illustrates an example graphical user interface of an application configured to remotely modify interfaces of a holistic processing application associated with symptoms according to aspects of the present disclosure. The process (or instance) of the holistic processing application executing on the server may include graphical user interface 800. Graphical user interface 800 includes box 804 with a set of ailments or conditions that may be affecting the user. Box 804 can be filtered using a query entered via a search field. For example, upon receiving the query, box 804 may remove entries that do not correspond to the query and leaving entries that do correspond to the query. The query may be based on the spelling of the entry or by the keywords associated with entry as shown in box 808.

Selection of a particular ailment or condition may populate box 808 with one or more comma-separated keywords usable to search for a selected ailment or condition. The keywords may include the ailment or condition, misspelled versions of the ailment or condition, symptoms associated with the ailment or condition, symptoms associated with related ailments or conditions, related ailments or conditions, comorbidities of the selected ailment or condition, etc. Box 808 may be modified by adding, removing, or editing keywords the keywords. Upon saving the selection, users operating the holistic processing application on computing devices, can search for ailments or conditions using the modified keywords.

For example, a user operating a holistic processing application may execute a query for a particular ailment or condition using one or more symptom keywords. The holistic processing application may execute the query using a local database and/or a remote database (e.g., associated with the process or instance of the holistic processing application executing on the server, etc.). In some instances, the holistic processing application may execute the query against the remote database before the local database because the remote database may be updated more frequently and therefore have more relevant information. Since query is executed against the remote database first, the keywords for each ailment or condition can be updated without affecting the operations for the holistic processing application executing on the computing device of the user. The process or instances of the holistic processing application executing on the server may return the results from the remote database along with instructions for presenting the results.

Graphical user interface 800 may also include fields that can modify when modifications to ailments or conditions can be implemented, when modifications to ailments or conditions can expire (e.g., causing the ailment or condition to be removed and/or the modification to the ailment or condition to be removed, etc.), the name of the ailment or condition, identifiers associated with ailment or condition, characteristics or properties of the ailment or condition, etc.

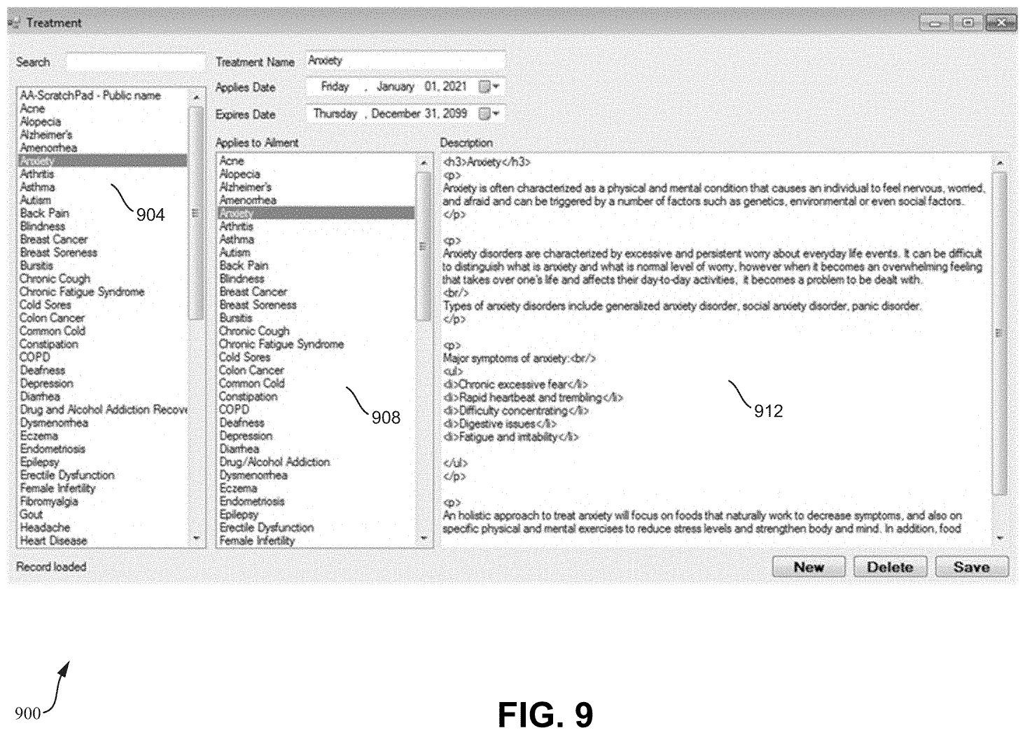

FIG. 9 illustrates an example graphical user interface of an application configured to remotely modify interfaces of a holistic processing application associated with treatment processes according to aspects of the present disclosure. The process (or instance) of the holistic processing application executing on the server may include graphical user interface 900. Graphical user interface 900 can be operated to dynamically modify treatment protocols of a holistic processing application executing on computing devices. Graphical user interface 900 includes box 904 identifying a set of treatment protocols that can be executed by the user. Box 904 can be filtered using a query entered via a search field. For example, upon receiving the query, box 904 may remove entries that do not correspond to the query and leaving entries that do correspond to the query. The query may be based on the spelling of the entry or by keywords associated with an entry (e.g., such as, but not limited to, ailments or conditions treated by the treatment protocol, symptoms associated with the treatment protocol, etc.).

Selection of a particular treatment protocol may populate box 908 with an identification of one or more ailments or conditions that may be treated with the selected treatment protocol. The one or more ailments or conditions may be modified by adding ailments or conditions, removing ailments or conditions, or modifying ailments or conditions. For example, selecting an ailment or condition in box 908 may populate box 912 with a sequence of instructions that can be executed by the holistic processing application executing on the computing device. The sequence of instructions may be executed by the holistic processing application executing on the computing device to, for example, provide a presentation of instructions that can be executed by the user to treat an ailment or condition, provide a presentation of instructions that can be executed by the user to treat one or more symptoms, present and/or modify user interfaces presented by the holistic processing application executing on the computing device, execute functions of the holistic processing application executing on the computing device, present information associated with the selected ailment or condition (e.g., description of the ailment or conditions, symptoms, treatment goals, etc.), etc. The sequence of instructions may include instructions configured to control the presentation, color, format, etc. of information associated with the selected ailment or condition.

Graphical user interface 900 may also include fields that can modify when the modification to the treatment protocol is to be effective, when modifications to the treatment protocol is to expire (e.g., causing the treatment protocol to be removed and/or the modification to the treatment protocol is to be removed, etc.), the name of the treatment protocol, identifiers associated with treatment protocol, characteristics or properties of the treatment protocol, an identification of ailments or conditions for which the treatment protocol may be selected, the set of instructions, etc.

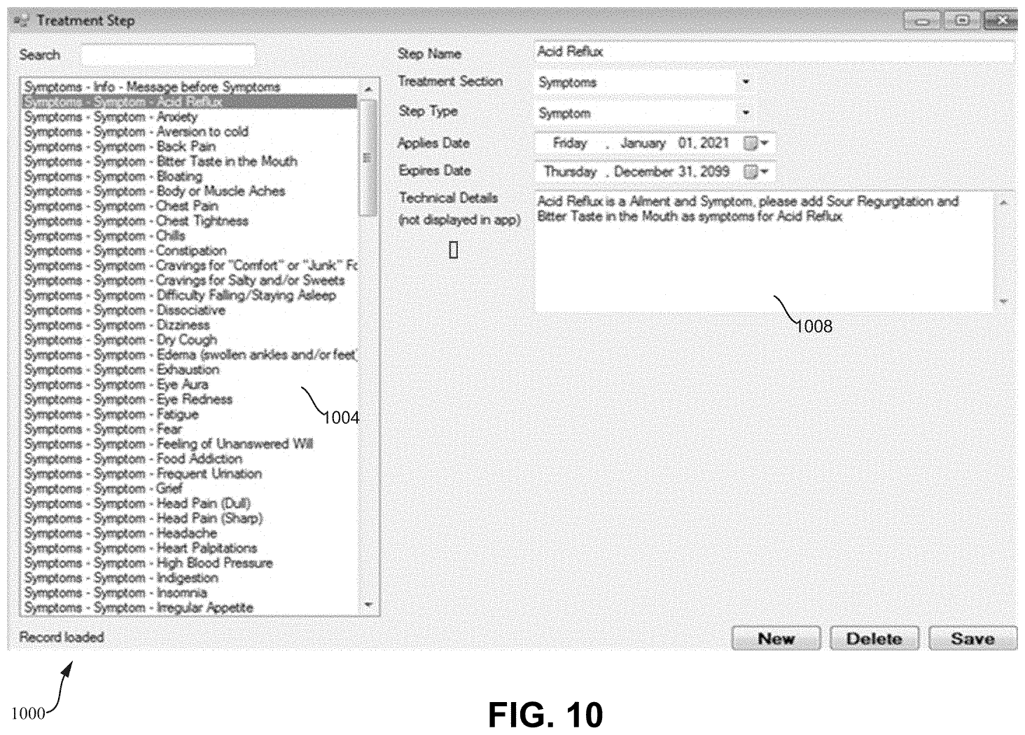

FIG. 10 illustrates another example graphical user interface of an application configured to remotely modify interfaces of a holistic processing application associated with treatment processes according to aspects of the present disclosure. The process (or instance) of the holistic processing application executing on the server may include graphical user interface 1000. Graphical user interface 1000 can be operated to dynamically modify symptoms of a holistic processing application executing on computing devices. The holistic processing application may enable selection of a set of symptoms from a predefined list of symptoms. The predefined list of symptoms may be defined by the process (or instance) of the holistic processing application executing on the server and be dynamically updated at any time causing a real time (e.g., approximately immediate) corresponding change at the holistic processing application executing on the computing device.

For example, box 1004 may include the predefined list of symptoms. Graphical user interface 1000 may enable adding new symptoms to the predefined list of symptoms, removing symptoms to the predefined list of symptoms, and/or editing symptom of the predefined list of symptoms. Entries in box 1004 may be selected causing additional information associated with the entry to be displayed in box 1008. The additional information may include a description of the selected symptom, technical details, ailments or conditions associated with the symptom, treatment protocols associated with the symptom, etc. The additional information may be modified (e.g., by adding, removing, and/or editing the content of box 1008).

Graphical user interface 1000 may also include fields that can modify when the modification to the symptom is to be effective, when modifications to symptom is to expire (e.g., causing the symptom to be removed and/or the modification to symptom is to be removed, etc.), the name of the symptom, identifiers associated with symptom, characteristics or properties of the symptom, an identification of ailments or conditions associated with the symptom, etc.

When a holistic processing application executing on the computing device executes an operation associated with a symptom (e.g., such as, but not limited to, presenting an interface associated with symptoms, generating or executing treatment protocols, requesting updated information from the user such as a health status, etc.), the holistic processing application may first update the predefined list of symptoms and corresponding additional information from the process (or instance) of the holistic processing application executing on the server. Alternatively, the holistic processing application may receive an updated predefined list of symptoms and/or additional information associated therewith each time the holistic processing application executes. The user may select one or more symptoms from the predefined list of symptoms from the holistic processing application to present the additional information (or a portion thereof), generate holistic treatment processes or treatment protocols, provide a health status (e.g., symptoms that are currently affecting the user, etc.).

The process (or instance) of the holistic processing application executing on the server may update the holistic processing application at runtime (e.g., while both the process (or instance) of the holistic processing application is executing on the server and the holistic processing application is executing on the computing device) without interrupting the operations of the holistic processing application that is executing on the computing device.

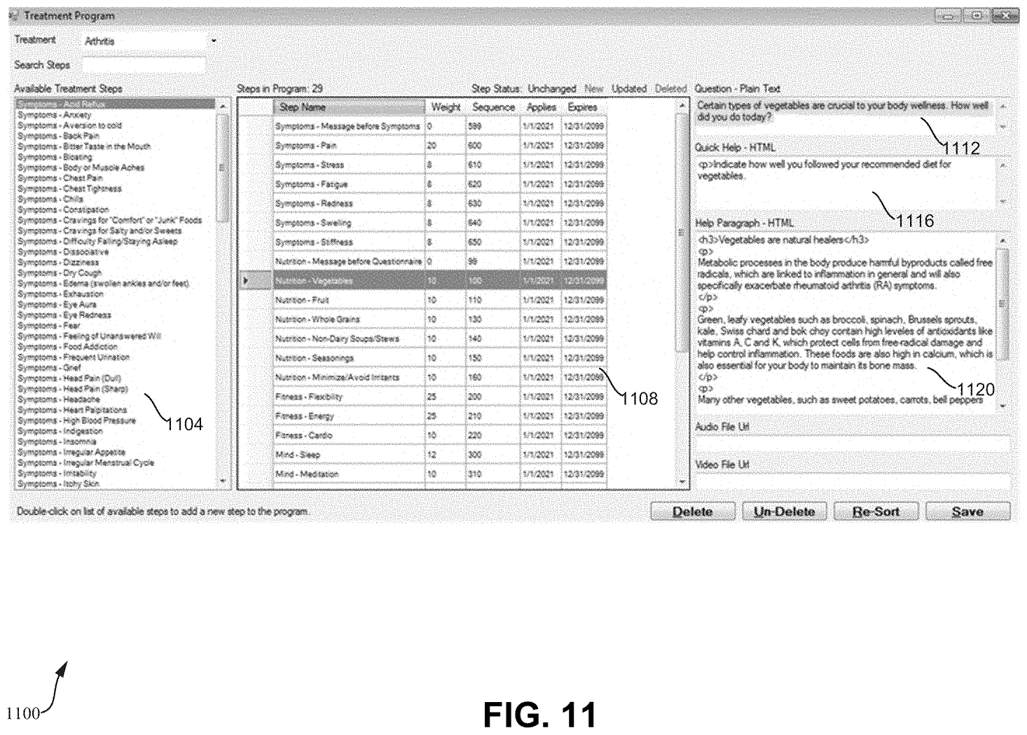

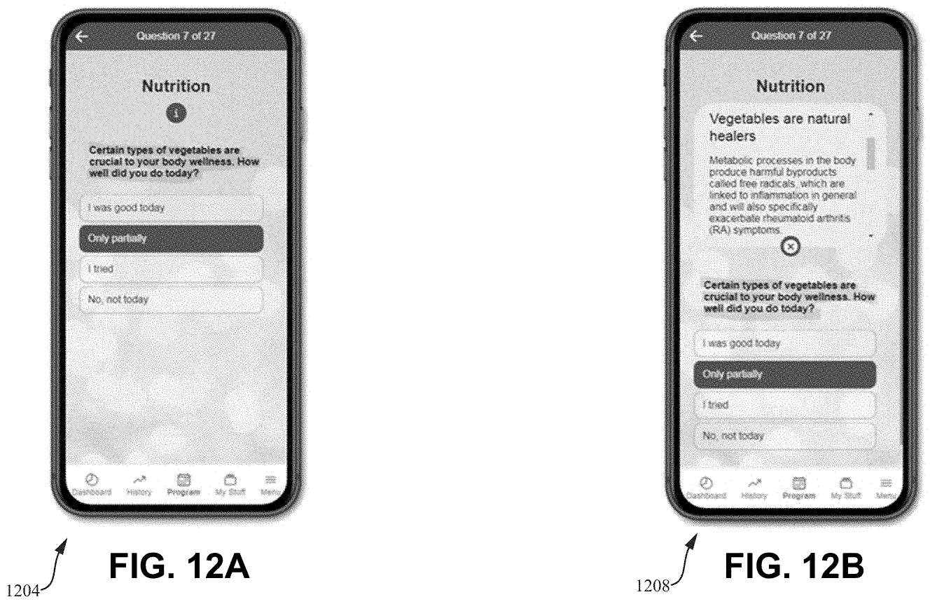

FIG. 11 illustrates another example graphical user interface of an application configured to remotely modify interfaces of a holistic processing application defining treatment processes according to aspects of the present disclosure. The process (or instance) of the holistic processing application executing on the server may include graphical user interface 1100. Graphical user interface 1100 can be operated to dynamically modify treatment protocols of a holistic processing application executing on computing devices. Graphical user interface 1100 may include box 1104 that includes an identification of treatment protocols associated with symptoms (of the predefined list of symptoms). The treatment protocols may include a sequence of steps that may be executed to alleviate the corresponding symptom.

Upon selecting a treatment protocol from box 1104, box 1108 may be populated with the sequence of steps corresponding to the selected treatment protocol. The sequence of steps may include a step identifier (e.g., such as a step name, etc.), a weight (e.g., a score assigned to the step indicate of a degree in which the step may affect alleviation of the corresponding symptom, etc.), a sequence (e.g., indicating a position of the step relative to other steps), an initiation date (e.g., indicative of when the step was generated, etc.), an expiration date (e.g., indicative of when the step will be deprecated, etc.). Steps may be added or removed from the sequence of the steps. The sequence of steps may be reordered.

In some instances, the sequence of steps may include one or more subsequences associated with each interdependent holistic class. For example, the sequence of steps may include one or more steps for each interdependent holistic class. The one or more subsequences may be executed in series with other subsequences or in parallel with other subsequences. The sequence of steps may also include one or more steps configured to augment one or more other steps. For instance, the sequence of steps may include steps configured to assess a current status of symptoms associated with the treatment protocol. The assessment may be based on responses to questions or prompts (e.g., text and/or audio, etc.), input from sensors (e.g., such as from a wearable device, etc.), or the like. The assessment of the one or more symptoms may cause a modification of a subsequent step. For instances, an assessment step of a pain symptom indicating an increase in joint pain may result in a modified exercise step.

Selection of a particular step may populate additional information associated with the step in box 1112, box 1116, and 1120. The additional information may include, but is not limited to, a description of the step, instructions to be executed (e.g., by the user, the holistic processing application, wearable device, etc.), audio segments associated with the step, video segments associated with the step, images associated with the step, and/or the like. For example, box 1112 includes general description of the step along a request for information from the user, box 1116 may be an encoded brief description of the step. The description may be encoded using hypertext transfer protocol (e.g., HTTP as shown) or other programming language that can be executed by the holistic processing application. Box 1120 may include encoded detailed information associated with the step. The detailed description may be encoded using HTTP (e.g., as shown) or other programming language configured to be executed by the holistic processing application. Box 1112, box 1116, and 1120 may be modified by adding, removing, or editing the text of the respective box 1112, box 1116, and 1120.

When the holistic processing application executes, the holistic processing application may determine if there is a holistic treatment process or treatment profile in progress. If so, the holistic processing application may retrieve the steps scheduled for execution within the next couple of hours (e.g., such as the within the current day) or alternatively all of the steps from process (or instance) of the holistic processing application executing on the server. If any of the steps changed since the holistic processing application last executed, the process (or instance) of the holistic processing application executing on the server may determine whether to transmit the original version of the steps (e.g., the version of the step that existed when the holistic treatment process or treatment protocol was generated) or the updated version. In some instances, the process (or instance) of the holistic processing application executing on the server may transmit the updated version. In other instances, the process (or instance) of the holistic processing application executing on the server may determine the degree in which the updated steps may affect other steps of the holistic treatment process or treatment protocol. If the degree in which the updated steps may affect other steps of the holistic treatment process or treatment protocol is less than a threshold, then the process (or instance) of the holistic processing application executing on the server may transmit the updated steps. If the degree in which the updated steps may affect other steps of the holistic treatment process or treatment protocol is greater than the threshold, then the process (or instance) of the holistic processing application executing on the server may transmit the original steps.

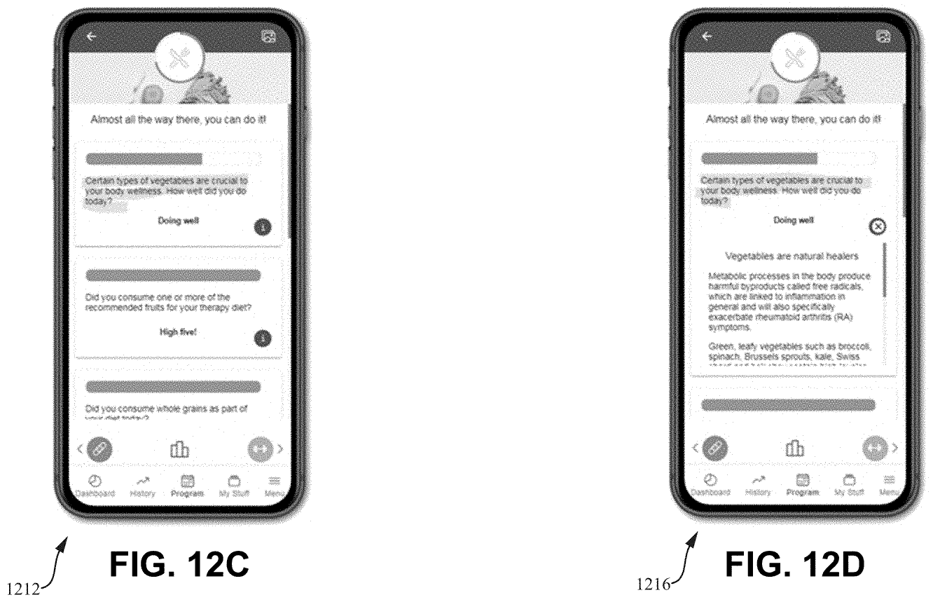

FIGS. 12A, 12B. 12C, and 12D illustrate example graphical user interfaces of a holistic processing application dynamically defined based remotely received instructions according to aspects of the present disclosure. Graphical user interface 1204 of FIG. 12A illustrates an assessment step of a treatment protocol requesting information associated with the nutrition interdependent holistic class. The treatment protocol may include any number of assessment steps. In some instances, input received in response to an assessment step may determine which assessment steps or other steps of the treatment protocol are to be executed. Graphical user interface 1208 of FIG. 12B includes an alternative representation of graphical user interface 1204 in which additional information associated with the assessment step may be dynamically presented. For instance, if the assessment step is modified (e.g., as described in connection to FIG. 11) while graphical user interface 1204 is being presented, the holistic processing application may automatically begin presenting graphical user interface 1208 allowing for real time modification of the holistic processing application. Alternatively, or additionally, Graphical user interface 1204 may include one or more hidden triggers that may be activated upon selection (e.g., such as by touching a particular area of a touchscreen, a gesture via the touchscreen, a particular sound or word, etc.). Once activated, the hidden trigger may cause the holistic processing application to switch from presenting graphical user interface 1204 to presenting graphical user interface 1208. The hidden trigger may be defined so as to assist the user to execute the assessment step (e.g., by providing additional information associated with the query of the assessment step, the possible responses to the query, previous or subsequent assessment steps, the treatment protocol, the interdependent holistic class, etc.).

Graphical user interface 1212 of FIG. 12C includes a presentation of results of execution of one or more assessment steps. In some instances, the results may be further based on the results of assessment steps of one or more previous treatment protocols. For each step, the graphical user interface 1212 may provide an indication of a degree in which the response likely positively or negatively impacted execution of the treatment protocol. The degree may be represented numerically (e.g., as an integer, percentage, fraction, etc.), graphically (e.g., as a progress bar, progress ring, etc.), or the like. Graphical user interface 1212 may provide additional information associated with the assessment step or the treatment protocol (e.g., such as a description of the assessment step, positive reinforcement associated with the response, etc.). Graphical user interface 1212 may also include one or more buttons configured to present a new graphical user interface with historical information (e.g., such as graph of responses to the same or similar assessment step over time, scores associated with interdependent holistic classes, indication of the contribution of a response to the assessment step to a score of the interdependent holistic class, etc.

Graphical user interface 1216 of FIG. 12D includes a representation of graphical user interface 1212 when a particular response of an assessment step is selected. Upon selection, holistic processing application may present additional information associated with the treatment process such as the information of box 1120 of FIG. 11. In some instances, when the particular response of an assessment step is selected, the holistic processing application may obtain the additional information of box 1120 that corresponds to the assessment step so as to present the most recent version of the additional information.

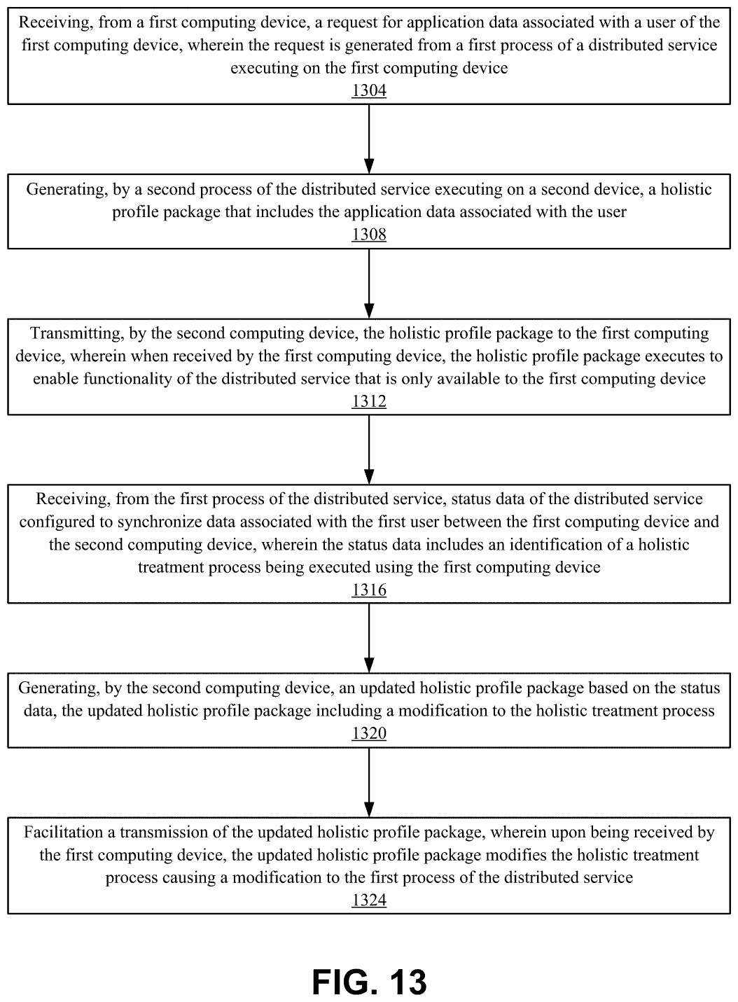

FIG. 13 illustrates a flowchart of an example process for generating holistic treatment processes executable to improve an overall wellness of a particular user according to aspects of the present disclosure. At block 1304, a server may receive, from a first computing device, a request for application data associated with a user of the first computing device. The request may be generated from a first process of a distributed service executing on the first computing device. For example, a holistic processing application (e.g., distributed service) may include one or more processes that may be executed by each of one or more devices. A first one or more processes may be executed by the computing device operated by a user. A second one or more process may be executed by the server that manages the first one or more processes executed by a many computing devices. The portion of the distrusted service configured to be executed by the computing device may generate the request for application data when first executing, upon detecting an event, in regular time intervals, and/or the like.

In some instances, the application data may be associated with a particular user (e.g., include holistic treatment processes generated for the particular user, symptom data associated with the particular user, etc.). The server may use information included in the request to determine an identification of the user and/or to authenticate the request. For example, the request may include an identification of the particular user, access credentials (e.g., username/password, etc.), a token associated with the particular user, etc.)

At block 1308, the server (e.g., a second computing device executing a second process of the distributed service) may generate a holistic profile package that includes the application data associated with the user. The application data may include information associated with the particular user and the distributed service that enables the distributed service to operate on the computing device. For example, the application data may include interfaces (e.g., graphical user interfaces, application programming interfaces, etc.), data associated with a user profile of the particular user, functions to be executed by the computing devices to provide functionality of the distributed service, etc. In some examples, the distributed service may store minimal information within memory of the computing device (e.g., such as user agnostic information, basic functionality of the portion of the distributed service that is to be executed by the computing device, etc.). Upon execution, the first process may request the application data configured to enable full functionality of the portion of the distributed service that is to be executed by the computing device (e.g., such as user specific data, interfaces, functions, etc.).

In some instances, the holistic profile package may be encrypted (e.g., using a hashing function, or the like) using a seed or key associated with the particular user or information associated with the particular user. The information may be further protected by salting the encryption algorithm to further protect the holistic profile package. The holistic profile package so as to prevent other users (or other computing devices) from accessing the holistic profile package if the holistic profile package is transmitted to the wrong device or intercepted.

At block 1312, the server may transmit the holistic profile package to the first computing device. When the holistic profile package is received by the first computing device, the holistic profile package may be executed (e.g., by the first process or another process) to enable functionality of the distributed service that may be limited to the first computing device. For example, the holistic profile package may include user-specific data and functionality that corresponds to the particular user. Other users that may receive holistic profile packages may receive different application data (e.g., associated with other respective users) to prevent personal identifiable information or sensitive health information of the particular user from be accessible by users other than the particular user

At block 1316, the server may receive, from the first process of the distributed service, status data of the distributed service configured to synchronize data associated with the first user between the first computing device and the second computing device. The server (e.g., second computing device) may store a duplicate of the information stored in memory of the computing device. Alternatively, the server may store a version of the information stored in memory of the computing device with less sensitive data (e.g., such as particular user data, PII, etc.). The first computing device may transmit data to the server to synchronize the data in regular time intervals, upon detecting an event (e.g., such as a change in data to be synchronized, etc.), user input, a request to terminate the first process, etc. The status data may include an identification of a holistic treatment process being executed using the first computing device.

At block 1320, the server may generate an updated holistic profile package based on the status data. The updated holistic profile package may include a modification to the holistic treatment process. The server may dynamically update the portion of the distributed service operating on the computing device in real time (e.g., as the portion of the distributed service operating on the computing device executes) and without requiring the first process to be terminated or restarted. The second process of the distributed service may determine, based on the status data, if there any updates to the treatment protocols, steps, symptoms, interfaces, functions, information, etc. associated with the holistic treatment process. If there are updates to any of the data associated with the holistic treatment process, then the server may generate the updated holistic profile package to dynamically update the holistic treatment.

At block 1324, the server may facilitate a transmission of the updated holistic profile package to the computing device. When the updated holistic profile package is received by the first computing device, the updated holistic profile package may update the holistic treatment process causing a modification to the first process of the distributed service. For example, the updating the holistic treatment process may modify interfaces (e.g., graphical user interfaces, application programming interfaces, etc.), information to be presented, functions of the distributed service, etc. The first process may be modified to increase the efficiency of operating the portion of the distributed service operating on the computing device (rearranging user interfaces, removing portions of interfaces that are unused or rarely used, changing the size or location of portions of interfaces that are frequently used, etc.), removing data or functions that are rarely executed, etc.

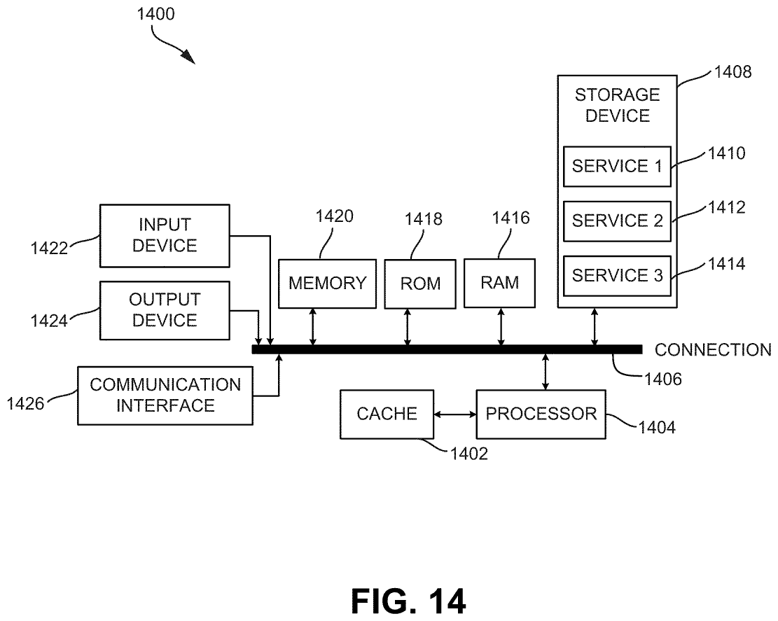

FIG. 14 illustrates an example computing device according to aspects of the present disclosure. For example, computing device 1400 can implement any of the systems or methods described herein. In some instances, computing device 1400 may be a component of or included within a media device. The components of computing device 1400 are shown in electrical communication with each other using connection 1406, such as a bus. The example computing device architecture 1400 includes a processor (e.g., CPU, processor, or the like) 1404 and connection 1406 (e.g., such as a bus, or the like) that is configured to couple components of computing device 1400 such as, but not limited to, memory 1420, read only memory (ROM) 1418, random access memory (RAM) 1416, and/or storage device 1408, to processing unit 1410.

Computing device 1400 can include a cache 1402 of high-speed memory connected directly with, in close proximity to, or integrated within processor 1404. Computing device 1400 can copy data from memory 1420 and/or storage device 1408 to cache 1402 for quicker access by processor 1404. In this way, cache 1402 may provide a performance boost that avoids delays while processor 1404 waits for data. Alternatively, processor 1404 may access data directly from memory 1420, ROM 817, RAM 1416, and/or storage device 1408. Memory 1420 can include multiple types of homogenous or heterogeneous memory (e.g., such as, but not limited to, magnetic, optical, solid-state, etc.).

Storage device 1408 may include one or more non-transitory computer-readable media such as volatile and/or non-volatile memories. A non-transitory computer-readable medium can store instructions and/or data accessible by computing device 1400. Non-transitory computer-readable media can include, but is not limited to magnetic cassettes, hard-disk drives (HDD), flash memory, solid state memory devices, digital versatile disks, cartridges, compact discs, random access memories (RAMs) 1425, read only memory (ROM) 1420, combinations thereof, or the like.

Storage device 1408, may store one or more services, such as service 1 1410, service 2 1412, and service 3 1414, that are executable by processor 1404 and/or other electronic hardware. The one or more services include instructions executable by processor 1404 to: perform operations such as any of the techniques, steps, processes, blocks, and/or operations described herein; control the operations of a device in communication with computing device 1400; control the operations of processing unit 1410 and/or any special-purpose processors; combinations therefor; or the like. Processor 1404 may be a system on a chip (SOC) that includes one or more cores or processors, a bus, memories, clock, memory controller, cache, other processor components, and/or the like. A multi-core processor may be symmetric or asymmetric.

Computing device 1400 may include one or more input devices 1422 that may represent any number of input mechanisms, such as a microphone, a touch-sensitive screen for graphical input, keyboard, mouse, motion input, speech, media devices, sensors, combinations thereof, or the like. Computing device 1400 may include one or more output devices 1424 that output data to a user. Such output devices 1424 may include, but are not limited to, a media device, projector, television, speakers, combinations thereof, or the like. In some instances, multimodal computing devices can enable a user to provide multiple types of input to communicate with computing device 1400. Communications interface 1426 may be configured to manage user input and computing device output. Communications interface 1426 may also be configured to managing communications with remote devices (e.g., establishing connection, receiving/transmitting communications, etc.) over one or more communication protocols and/or over one or more communication media (e.g., wired, wireless, etc.).

Computing device 1400 is not limited to the components as shown if FIG. 14. Computing device 1400 may include other components not shown and/or components shown may be omitted.

The following examples illustrate various aspects of the present disclosure. As used below, any reference to a series of examples is to be understood as a reference to each of those examples disjunctively (e.g., “Examples 1-4” is to be understood as “Examples 1, 2, 3, or 4”).

Example 1 is a computer-implemented method comprising: receiving an identification of one or more symptoms, the one or more symptoms being associated with a user profile; executing a machine-learning model using the identification of the one or more symptoms and the user profile, the machine-learning model being configured to generate a holistic treatment process, wherein the holistic treatment process is configured to alleviate the one or more symptoms when executed by a user, and wherein the holistic treatment process includes treatment protocols for a set of interdependent holistic classes; facilitating a presentation of the holistic treatment process; receiving performance data corresponding to execution of the holistic treatment process over a first time interval; modifying the machine-learning model using the performance data to generate an updated machine-learning model, wherein the updated machine-learning model is configured to generate a revised holistic treatment process that is more likely to alleviate the one or more symptom; and facilitating a presentation of the revised holistic treatment process, wherein the revised holistic treatment process, when executed by the user, increases a likelihood of alleviating the one or more symptoms.

Example 2 is the computer-implemented method of example 1, wherein the first time interval is dynamically defined based on the performance data and an accuracy metric associated with the machine-learning model.

Example 3 is the computer-implemented method of any of example(s) 1-2, wherein the set of interdependent holistic classes include one or more of: a treatment class, a food class, a mind class, a supplement class, and a fitness class.

Example 4 is the computer-implemented method of any of example(s) 1-3, wherein presenting the holistic treatment process includes presenting a tutorial corresponding to the treatment protocols.

Example 5 is the computer-implemented method of any of example(s) 1-4, wherein a portion of the performance data associated with a particular interdependent holistic class is received from a remote device, wherein the remote device hosts an application that corresponds to the particular interdependent holistic class.

Example 6 is the computer-implemented method of any of example(s) 1-5, further comprising: receiving, after an expiration of the first time interval, feedback corresponding to the holistic treatment process from the user and at least one user device, wherein the feedback includes an indication as to whether the one or more symptoms have been alleviated; and training the machine-learning model using reinforcement learning based on the feedback, wherein training the machine-learning model improves a subsequent holistic treatment process generated for the user.

Example 7 is the computer-implemented method of any of example(s) 1-6, further comprising: generating, by the machine-learning model using the user profile, a value for each interdependent holistic class, wherein the value represents a degree of user wellness in the interdependent holistic class; generating a first user interface including a representation of each interdependent holistic class of the set of interdependent holistic classes, wherein the representation of each interdependent holistic class is based on the value associated with that interdependent holistic class; and presenting the first user interface.

Example 8 is the computer-implemented method of any of example(s) 1-7, further comprising: receiving input selecting a particular representation of a particular interdependent holistic class; generating a second user interface including an identification of the value associated with the particular interdependent holistic class and one or metrics corresponding to features used by the machine-learning model to generate the value for the particular interdependent holistic class; and presenting the second user interface.

Example 9 is the computer-implemented method of any of example(s) 1-8, further comprising: determining a weight for each interdependent holistic class based on one or more holistic treatment processes generated for the user, the weights indicating a degree of codependence of an interdependent holistic class on one or more other interdependent holistic classes; generating an overall value for the user, the overall value being a weighted sum of the values of each interdependent holistic class; and presenting the overall value.

Example 10 is a computer-implemented method comprising: receiving, from a first computing device, a request for application data associated with a user of the first computing device, wherein the request is generated from a first process of a distributed service executing on the first computing device; generating, by a second process of the distributed service executing on a second device, a holistic profile package that includes the application data associated with the user; transmitting, by the second computing device, the holistic profile package to the first computing device; wherein when received by the first computing device, the holistic profile package executes to enable functionality of the distributed service that is only available to the first computing device; receiving, from the first process of the distributed service, status data of the distributed service configured to synchronize data associated with the first user between the first computing device and the second computing device, wherein the status data includes an identification a holistic treatment being executed using the first computing device; generating, by the second computing device, an updated holistic profile package based on the status data, the updated holistic profile package including a modification to the holistic treatment process; and facilitation a transmission of the updated holistic profile package, wherein upon being received by the first computing device, the updated holistic profile package modifies the holistic treatment process causing a modification to the first process of the distributed service.

Example 11 is the computer-implemented method of example 10, wherein the modification to the holistic treatment process includes replacing a treatment protocol with different treatment protocol.

Example 12 is the computer-implemented method of any of example(s) 1-11, wherein the modification to the holistic treatment process includes adding a new treatment protocol.

Example 13 is the computer-implemented method of any of example(s) 1-12, wherein the modification to the holistic treatment process includes removing a new treatment protocol.

Example 14 is the computer-implemented method of any of example(s) 1-13, wherein the modification to the holistic treatment process includes reordering a sequence in which treatment protocols are to be executed.

Example 15 is the computer-implemented method of any of example(s) 1-14, wherein the modification to the first process of the distributed service alters one or more user interfaces of the distributed service.

Example 16 is the computer-implemented method of any of example(s) 1-15, wherein the status data includes one or more customizations to the holistic treatment process being executed using the first computing device, wherein the one or more customizations modify a process for generating new holistic treatment processes, and wherein the one or more customizations are stored separately from other data of the distributed service.

Example 17 is a system comprising: one or more processors; and a non-transitory computer-readable medium storing instructions that when executed by the one or more processors cause the one or more processor to perform any of example(s) 1-16.

Example 18 is a non-transitory computer-readable medium storing instructions that when executed by one or more processors, cause the one or more processor to perform any of example(s) 1-16.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored in a form that excludes carrier waves and/or electronic signals. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

Some portions of this description describe examples in terms of algorithms and symbolic representations of operations on information. These operations, while described functionally, computationally, or logically, may be implemented by computer programs or equivalent electrical circuits, microcode, or the like. Furthermore, arrangements of operations may be referred to as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module can be implemented with a computer-readable medium storing computer program code, which can be executed by a processor for performing any or all of the steps, operations, or processes described.

Some examples may relate to an apparatus or system for performing any or all of the steps, operations, or processes described. The apparatus or system may be specially constructed for the required purposes, and/or it may comprise a general-purpose computing device selectively activated or reconfigured by a computer program stored in memory of computing device. The memory may be or include a non-transitory, tangible computer readable storage medium, or any type of media suitable for storing electronic instructions, which may be coupled to a bus. Furthermore, any computing systems referred to in the specification may include a single processor or multiple processors.

While the present subject matter has been described in detail with respect to specific examples, it will be appreciated that those skilled in the art, upon attaining an understanding of the foregoing, may readily produce alterations to, variations of, and equivalents to such embodiments. Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. Accordingly, the present disclosure has been presented for purposes of example rather than limitation, and does not preclude the inclusion of such modifications, variations, and/or additions to the present subject matter as would be readily apparent to one of ordinary skill in the art.

For clarity of explanation, in some instances the present disclosure may be presented as including individual functional blocks including functional blocks comprising devices, device components, steps or routines in a method embodied in software, or combinations of hardware and software. Additional functional blocks may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual examples may be described herein as a process or method which may be depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but may have additional steps not shown. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general-purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc.

Devices implementing the methods and systems described herein can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. The program code may be executed by a processor, which may include one or more processors, such as, but not limited to, one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A processor may be a microprocessor; conventional processor, controller, microcontroller, state machine, or the like. A processor may also be implemented as a combination of computing components (e.g., a combination of a DSP and a microprocessor, a plurality of microprocessors, one or more microprocessors in conjunction with a DSP core, or any other such configuration). Accordingly, the term “processor,” as used herein may refer to any of the foregoing structure, any combination of the foregoing structure, or any other structure or apparatus suitable for implementation of the techniques described herein. Functionality described herein also can be embodied in peripherals or add-in cards. Such functionality can also be implemented on a circuit board among different chips or different processes executing in a single device, by way of further example.

In the foregoing description, aspects of the disclosure are described with reference to specific examples thereof, but those skilled in the art will recognize that the disclosure is not limited thereto. Thus, while illustrative examples of the disclosure have been described in detail herein, it is to be understood that the inventive concepts may be otherwise variously embodied and employed, and that the appended claims are intended to be construed to include such variations. Various features and aspects of the above-described disclosure may be used individually or in any combination. Further, examples can be utilized in any number of environments and applications beyond those described herein without departing from the broader spirit and scope of the disclosure. The disclosure and figures are, accordingly, to be regarded as illustrative rather than restrictive.

The various illustrative logical blocks, modules, circuits, and algorithm steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or media devices of the computing platform. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method comprising:
receiving an identification of one or more symptoms, the one or more symptoms being associated with a user profile;
executing a machine-learning model using the identification of the one or more symptoms and the user profile, the machine-learning model being configured to generate a holistic treatment process, wherein the holistic treatment process is configured to alleviate the one or more symptoms when executed by a user, and wherein the holistic treatment process includes treatment protocols for a set of interdependent holistic classes;
facilitating a presentation of the holistic treatment process;
receiving performance data corresponding to execution of the holistic treatment process over a first time interval;
modifying the machine-learning model using the performance data to generate an updated machine-learning model, wherein the updated machine-learning model is configured to generate a revised holistic treatment process that is more likely to alleviate the one or more symptoms; and
facilitating a presentation of the revised holistic treatment process, wherein the revised holistic treatment process, when executed by the user, increases a likelihood of alleviating the one or more symptoms.

2. The computer-implemented method of claim 1, wherein the first time interval is dynamically defined based on the performance data and an accuracy metric associated with the machine-learning model.

3. The computer-implemented method of claim 1, wherein the set of interdependent holistic classes include one or more of: a treatment class, a food class, a mind class, a supplement class, and a fitness class.

4. The computer-implemented method of claim 1, wherein presenting the holistic treatment process includes presenting a tutorial corresponding to the treatment protocols.

5. The computer-implemented method of claim 1, wherein a portion of the performance data associated with a particular interdependent holistic class is received from a remote device, wherein the remote device hosts an application that corresponds to the particular interdependent holistic class.

6. The computer-implemented method of claim 1, further comprising:
receiving, after an expiration of the first time interval, feedback corresponding to the holistic treatment process from the user and at least one user device, wherein the feedback includes an indication as to whether the one or more symptoms have been alleviated; and
training the machine-learning model using reinforcement learning based on the feedback, wherein training the machine-learning model improves a subsequent holistic treatment process generated for the user.

7. The computer-implemented method of claim 1, further comprising:
generating, by the machine-learning model using the user profile, a value for each interdependent holistic class, wherein the value represents a degree of user wellness relative to the interdependent holistic class;
generating a first user interface including a representation of each interdependent holistic class of the set of interdependent holistic classes, wherein the representation of each interdependent holistic class is based on the value associated with that interdependent holistic class; and
presenting the first user interface.

8. A system comprising:
one or more processors; and
a non-transitory computer-readable medium storing instructions that when executed by the one or more processors cause the one or more processor to perform operations including:
receiving an identification of one or more symptoms, the one or more symptoms being associated with a user profile;
executing a machine-learning model using the identification of the one or more symptoms and the user profile, the machine-learning model being configured to generate a holistic treatment process, wherein the holistic treatment process is configured to alleviate the one or more symptoms when executed by a user, and wherein the holistic treatment process includes treatment protocols for a set of interdependent holistic classes;
facilitating a presentation of the holistic treatment process;
receiving performance data corresponding to execution of the holistic treatment process over a first time interval;
modifying the machine-learning model using the performance data to generate an updated machine-learning model, wherein the updated machine-learning model is configured to generate a revised holistic treatment process that is more likely to alleviate the one or more symptoms; and
facilitating a presentation of the revised holistic treatment process, wherein the revised holistic treatment process, when executed by the user, increases a likelihood of alleviating the one or more symptoms.

9. The system of claim 8, wherein the first time interval is dynamically defined based on the performance data and an accuracy metric associated with the machine-learning model.

10. The system of claim 8, wherein the set of interdependent holistic classes include one or more of: a treatment class, a food class, a mind class, a supplement class, and a fitness class.

11. The system of claim 8, wherein presenting the holistic treatment process includes presenting a tutorial corresponding to the treatment protocols.

12. The system of claim 8, wherein a portion of the performance data associated with a particular interdependent holistic class is received from a remote device, wherein the remote device hosts an application that corresponds to the particular interdependent holistic class.

13. The system of claim 8, wherein the operations further include:
receiving, after an expiration of the first time interval, feedback corresponding to the holistic treatment process from the user and at least one user device, wherein the feedback includes an indication as to whether the one or more symptoms have been alleviated; and
training the machine-learning model using reinforcement learning based on the feedback, wherein training the machine-learning model improves a subsequent holistic treatment process generated for the user.

14. The system of claim 8, wherein the operations further include:
generating, by the machine-learning model using the user profile, a value for each interdependent holistic class, wherein the value represents a degree of user wellness relative to the interdependent holistic class;
generating a first user interface including a representation of each interdependent holistic class of the set of interdependent holistic classes, wherein the representation of each interdependent holistic class is based on the value associated with that interdependent holistic class; and
presenting the first user interface.

15. A non-transitory computer-readable medium storing instructions that when executed by one or more processors, cause the one or more processor to perform operations including:
receiving an identification of one or more symptoms, the one or more symptoms being associated with a user profile;
executing a machine-learning model using the identification of the one or more symptoms and the user profile, the machine-learning model being configured to generate a holistic treatment process, wherein the holistic treatment process is configured to alleviate the one or more symptoms when executed by a user, and wherein the holistic treatment process includes treatment protocols for a set of interdependent holistic classes;
facilitating a presentation of the holistic treatment process;
receiving performance data corresponding to execution of the holistic treatment process over a first time interval;
modifying the machine-learning model using the performance data to generate an updated machine-learning model, wherein the updated machine-learning model is configured to generate a revised holistic treatment process that is more likely to alleviate the one or more symptoms; and
facilitating a presentation of the revised holistic treatment process, wherein the revised holistic treatment process, when executed by the user, increases a likelihood of alleviating the one or more symptoms.

16. The non-transitory computer-readable medium of claim 15, wherein the first time interval is dynamically defined based on the performance data and an accuracy metric associated with the machine-learning model.

17. The non-transitory computer-readable medium of claim 15, wherein the set of interdependent holistic classes include one or more of: a treatment class, a food class, a mind class, a supplement class, and a fitness class.

18. The non-transitory computer-readable medium of claim 15, wherein presenting the holistic treatment process includes presenting a tutorial corresponding to the treatment protocols.

19. The non-transitory computer-readable medium of claim 15, wherein a portion of the performance data associated with a particular interdependent holistic class is received from a remote device, wherein the remote device hosts an application that corresponds to the particular interdependent holistic class.

20. The non-transitory computer-readable medium of claim 15, wherein the operations further include:
receiving, after an expiration of the first time interval, feedback corresponding to the holistic treatment process from the user and at least one user device, wherein the feedback includes an indication as to whether the one or more symptoms have been alleviated; and
training the machine-learning model using reinforcement learning based on the feedback, wherein training the machine-learning model improves a subsequent holistic treatment process generated for the user.
